(19) United States
(12) Patent Application Publication
(10) Pub. No.: US 2003/0219410 A1
(43) Pub. Date: Nov. 27, 2003

(54) Adenoviral vectors for modulating the cellular activities associated to PODs

(75) Inventor: Manuel Rosa Calatrava, Strasbourg (FR)

Correspondence Address: BURNS DOANE SWECKER & MATHIS L L P, POST OFFICE BOX 1404, ALEXANDRIA, VA, 22313-1404, US

(73) Assignee: TRANSGENE S.A., STRASBOURG CEDEX (FR)

(21) Appl. No.: 10/355,277

(22) Filed: Jan. 31, 2003

Related U.S. Application Data
(60) Provisional application No. 60/353,226, filed on Feb. 4, 2002.

(30) Foreign Application Priority Data
Feb. 1, 2002 (EP) 02 36 0050.5

(51) Int. Cl. A61K 48/00; C12N 15/861

(52) U.S. Cl. 424/93.2; 435/456

(57) ABSTRACT

The present invention concerns a method of modulating one or more cellular activities dependent on a POD nuclear structure in a host cell through the action of a molecule of adenoviral origin, wherein said molecule of adenoviral origin is capable of interacting with the cellular function of said POD nuclear structure. In a first aspect, the present invention provides a method, a replication-defective adenoviral vector and a composition intended to reduce or inhibit one or more POD-dependent cellular activities by introducing said adenoviral molecule in the host cell. The invention also relates to the use of such replication-defective adenoviral vector or molecule to provide a reduction or an inhibition of the antiviral or apoptosis cellular activities as well as to provide a reduction of the toxicity induced by a replication-defective adenovirus vector or to enhance transgene expression driven from said replication-defective adenovirus vector. In a second aspect, the present invention provides a replication-competent adenoviral vector having native pIX or E4orf3 gene non-functional or deleted, as well as a viral particle, a host cell and a composition comprising such a replication-competent adenoviral vector and a method of treatment using such a replication-competent adenoviral vector. The present invention also concerns a method of enhancing apoptosis in a host cell using such a replication-competent adenoviral vector.

### CROSS-REFERENCE TO PRIORITY/PROVISIONAL APPLICATIONS

This application claims priority under 35 U.S.C. §119 of EP 02/36 0050.5, filed Feb. 1, 2002, and of provisional application Serial No. 60/353,226, filed Feb. 4, 2002, both hereby expressly incorporated by reference. The application is also a continuation of said '226 provisional.

### BACKGROUND OF THE INVENTION

1. Technical Field of the Invention

The present invention concerns a method of modulating one or more cellular activities dependent on a POD nuclear structure in a host cell through the action of a molecule of adenoviral origin, wherein said molecule of adenoviral origin is capable of interacting with the cellular function of said POD nuclear structure. In a first aspect, the present invention provides a method, a replication-defective adenoviral vector and a composition intended to reduce or inhibit the POD-dependent cellular activities by introducing said adenoviral molecule in the host cell. The invention also relates to the use of such replication-defective adenoviral vector or molecule to provide a reduction or an inhibition of the antiviral or cellular apoptosis activities as well as to provide a reduction of the toxicity induced by a replication-defective adenovirus vector or to enhance transgene expression driven from said replication-defective adenovirus vector. In a second aspect, the present invention provides a replication-competent adenoviral vector having native pIX or E4orf3 gene non-functional or deleted, as well as a viral particle, a host cell and a composition comprising such a replication-competent adenoviral vector and a method of treatment using such a replication-competent adenoviral vector. The present invention also concerns a method of enhancing apoptosis in a host cell using such a replication-competent adenoviral vector. The present invention is particularly useful in gene therapy to enhance the therapeutic effect of adenovirus gene therapy vectors.

2. Description of the Prior Art

Gene therapy can be defined as the transfer of genetic material into a cell or an organism. The possibility of treating human disorders by gene therapy has changed in the last few years from the stage of theoretical considerations to that of clinical applications. The first protocol applied to man was initiated in the USA in September 1990 on a patient suffering from adenine deaminase (ADA) deficiency. This first encouraging experiment has been followed by numerous new applications and promising clinical trials based on gene therapy are currently ongoing (see for example clinical trials listed at http://cnetdb.nci.nih.gov/trialsrch.shtml or http://www.wiley.co.uk/genetherapy/clinical/). The large majority of the current protocols employ vectors to carry the therapeutic gene to the cells to be treated.

There are two main types of gene-delivery vectors, viral and non-viral. Viral vectors are derived from naturally-occurring viruses and use the diverse and highly sophisticated mechanisms that wild-type viruses have developed to cross the cellular membrane, escape from lysosomal degradation and deliver their genome to the nucleus. Many different viruses are being adapted as vectors, but the most advanced are retrovirus, adenovirus and adeno-associated virus (AAV) (Robbins et al., Trends Biotechnol. 16 (1998), 35-40; Miller, Human Gene Therapy 8 (1997), 803-815; Montain et al., Tibtech 18 (2000), 119-128). Substantial effort has also gone into developing viral vectors based on poxviruses (especially vaccinia) and herpes simplex virus (HSV). Non-viral approaches include naked DNA (i.e., plasmidic DNA; Wolff et al., Science 247 (1990), 1465-1468), DNA complexed with cationic lipids (for a review see, for example, Rolland, Critical reviews in Therapeutic Drug Carrier Systems 15 (1998), 143-198) and particles comprising DNA condensed with cationic polymers (Wagner et al., Proc. Natl. Acad. Sci. USA 87 (1990), 3410-3414 and Gottschalk et al., Gene Ther. 3 (1996), 448-457). At the present stage of development, the viral vectors generally give the most efficient transfection but their main disadvantages include their limited cloning capacity, their tendency to elicit immune and inflammatory responses and their manufacturing difficulties. Non-viral vectors achieve less efficient transfection but have no insert-size limitation, are less immunogenic and easier to manufacture.

Adenoviruses have been detected in many animal species, are non-integrative and low pathogene. They are able to infect a variety of cell types, dividing as well as quiescent cells. They have a natural tropism for airway epithelia. In addition, they have been used as live enteric vaccines for many years with an excellent safety profile. Finally, they can be easily grown and purified in large quantities. These features have made adenoviruses particularly appropriate for use as gene therapy vectors for therapeutic and vaccine purposes.

All adenoviruses are morphologically and structurally similar. These viruses are non-enveloped, regular icosahedrons, 60-90 nm in diameter consisting of an external capsid and an internal core. The capsid is constituted of 252 capsomers arranged geometrically to form 240 hexons and 12 penton bases from which fibers protude.

Their genome consists of a linear double-standed DNA molecule of approximately 36 kb (conventionally divided into 100 map units (mu)) carrying more than about thirty genes necessary to complete the viral cycle. During productive adenoviral infection, three classes of viral genes are temporally expressed in the following order: early (E), intermediate and late (L). The early genes are divided into 4 regions dispersed in the adenoviral genome (E1 to E4). The E1, E2 and E4 regions are essential to viral replication, whereas the E3 region is dispensable in this respect. The E1 region (E1A and E1B) encodes proteins responsible for the regulation of transcription of the viral genome. Expression of the E2 region genes (E2A and E2B) leads to the synthesis of the polypeptides needed for viral replication (Pettersson and Roberts, In Cancer Cells (Vol 4), DNA Tumor Viruses (1986); Botchan and Glodzicker Sharp Eds., Cold Spring Harbor Laboratory, Cold Spring Harbor, N.Y., 37-47). The proteins encoded by the E3 region prevent cytolysis by cytotoxic T cells and tumor necrosis factor (Wold and Gooding, Virology 184 (1991), 1-8). The E4 proteins encoded by the E4 region are involved in DNA replication, late gene expression and splicing and host cell shut off (Halbert et al., J. virol. 56 (1985), 250-257). The late genes (L1 to L5) overlap at least in part with the early transcription units and encode in their majority the structural proteins constituting the viral capsid. The products of the late genes are expressed after processing of a 20 kb primary transcript driven by the major late promoter (MLP). In addition, the adenoviral genome carries at both extremities cis-acting regions essential for DNA replication, respectively the 5′ and 3′ ITRs (Inverted Terminal Repeats) which harbor origins of DNA replication and a packaging sequence.

The product of the adenoviral intermediate Ad2 or Ad5 gene IX encodes a polypeptide (pIX) of 140 amino acid residues the expression of which is dependent on viral replication. Moreover, pIX has multifunctional properties. It was known for years that pIX is a structural component of the viral capsid that contributes to its stability by ensuring optimal cohesion between hexons (Furcinitti et al., EMBO J. 8 (1989), 3563-3570). Furthermore, it is essential for packaging the full length adenoviral genome (Ghosh-Choudhury et al., EMBO J. 6 (1987), 1733). It has also been recently shown that pIX is a transcriptional activator of several viral and cellular TATA-containing promoters (Lutz et al., J. Virol. 71 (1997), 5102-5109). Finally, production of pIX in infected cells leads to the formation of specific nuclear structures, that have been named clear amorphous inclusions (c.a. inclusions), due to their relative density to electron transmission (Rosa-Calatrava et al., J. Virol. 75 (2000), 7131-7141), the function of which being to take part to the viral-induced reorganization of host nuclear ultrastructures.

Mutational analysis have allowed to precisely delimit the functional domains of the pIX protein. The highly conserved N-terminal part of the protein is essential for the capsidic structural properties whereas the C-terminal leucine repeat (putative coiled-coil domain) is critical for the trans-activation function. The integrity of the leucine repeat appears to be essential for the formation and nuclear retention of the ca inclusions, likely through multimerisation of pIX with itself or with specific nuclear components via its coiled-coil domain (Rosa-Calatrava et al., J. Virol. 75 (2001), 7131-7141).

The adenoviral vectors presently used in gene therapy protocols are replication-deficient viruses lacking the E1 region, to avoid their dissemination in the environment and the host organism. Moreover, most of the adenoviral vectors are also E3 deleted, in order to increase their cloning capacity. The feasibility of gene transfer using these vectors has been demonstrated into a variety of tissues in vivo (see, for example, Yei et al., Hum. Gene Ther. 5 (1994), 731-744; Dai et al., Proc. Natl. Acad. Sci. USA 92 (1995), 1401-1405; Howell et al., Hum. Gene Ther. 9 (1998), 629-634; Nielsen et al., Hum. Gene Ther. 9 (1998), 681-694; U.S. Pat. No. 6,099,831; U.S. Pat. No. 6,013,638). However, their use is associated with acute inflammation and toxicity in a number of animal models (Yang et al., Proc. Natl. Acad. Sci. USA 91 (1994), 4407-4411; Zsengeller et al., Hum. Gene Ther. 6 (1995), 457-467) as well as with host immune responses to the viral vector and gene products (Yang et al., J. Virol. 69 (1995), 2004-2015), resulting in the elimination of the infected cells and only a transient gene expression.

The success of most viral vector-based gene transfer strategies depends on absence of vector-mediated toxicity as well as efficient transgene expression, in particular in view of treatment of chronic and genetic diseases. A reduction of toxicity has been attempted by deleting viral gene functions in order to abolish the residual synthesis of the viral antigens which is postulated to be responsible for the stimulation of inflammatory responses (see for example EP-974,668, U.S. Pat. No. 5,670,488). The evaluation of E1 and E4-deleted adenoviral vectors in vivo have shown contradictory results with regard to transgene persistence (Dedieu et al., J. Virol. 71 (1997), 4626-4637; Kaplan et al., Hum. Gene Ther. 8 (1997), 45-56; Armentano et al., J. Virol. 71 (1997), 2408-2416) although a reduced hepatotoxicity and inflammation was observed (Christ et al., Human Gene Ther. 11 (2000), 415-427).

Specific nuclear structures designated PODs (PML Oncogenic domains) or ND10 or PML nuclear domains were found to be associated with the nuclear matrix (for review, see, Doucas and Evans, Biochem. Biophys. Acta 1288 (1996), M25-9; Hodges et al., Am. J. Hum. Genet. 63 (1998), 297-304). Their size and number vary according to the type and the stage of the cellular cycle. Several proteins associated with the POD structures have been identified, including PML (Promyelocytic Leukemia Protein) which constitutes the organizer of POD structures, SP100 (Speckled 100 kDa), SUMO as well as various cellular factors involved in replication, transcription, chromosome modeling or apoptosis (see, Negorev and Maul, Oncogene 20 (2001), 7234-7242). Based on these observations, the nuclear structures of PODs are thought to be involved in the regulation of various cellular processes, including cell growth (Everett et al., J. Cell. Sci. 112 (1999), 4581-4588), differentiation (Wang et al., Science 279 (1998), 1547-1551) and apoptosis (Quignon et al., Nat. Genet. 20 (1998), 259-265). They have been shown to also participate to cellular antiviral processes (Chelbi-Alix et al., Leukemia 9 (1995), 2027-2033; Chelbi-Alix et al., J. Virol. 72 (1996), 1043-1051). In this context, several studies have documented the targeting of viral genomes to PODs and the disruption of PML-containing nuclear structures by viral regulatory proteins (see, for example, Everett, Oncogene 20 (2001), 7266-7273). One hypothesis is that PODs represent a cellular compartment repressive for viral gene expression, as several POD protein components are functionally linked the cellular interferon pathway. On this basis, it has been presumed that the disruption of the POD may be a virus-mediated mechanism to escape a cellular antiviral response and would therefore be a necessary early event in the replication cycle of many viruses to allow efficient expression of viral genes.

With respect to adenovirus, it has been shown that the Ad2 or Ad5 viral product E4orf3 induces the redistribution of PML protein from PODs to viral “fibrous-like” structures, during the early phase of infection (Carvalho et al., J. Cell Biol. 131 (1995), 45-56; Doucas et al., Genes Dev. 10 (1996), 196-207), thus inducing POD disruption. However, so far it has not been found conceivable in the prior art that the interaction of adenoviral molecules with POD nuclear structures could be a starting point for developing less toxic and, with respect to transgene expression, more efficient adenoviral vectors.

In view of the above-described prevalent difficulties associated with the use of adenoviral vectors in gene therapy, in particular concerning toxicity, transiency of transgene expression and antigenic effects, it is the problem underlying the present invention to improve the therapeutic benefit of gene therapy vectos.

### SUMMARY OF THE INVENTION

This problem is solved by the provision of the embodiments characterized in the claims.

Accordingly, the present invention relates to a method of modulating one or more cellular activitie(s) dependent on POD nuclear structure in a host cell through the action of a molecular of adenoviral origin, wherein said molecular of adenoviral origin is capable of interacting with the cellular function of said POD nuclear structure. Preferably, said action of a molecule of adenoviral origin is accomplished by contacting said molecule with said POD nuclear structure, wherein said molecule is capable of interacting with said POD nuclear structure.

The present invention is based on experiments in which it was shown that the adenoviral protein pIX probably takes part at the adenovirus-mediated alteration of PODs by redistributing the PML protein into c.a. inclusions during the late phase of infection, thereby neutralizing the function of this protein. This has the effectof a permanent disruption of PODs during the course of infection, potentially contributing to an optimal viral proliferation. Immunogold labeling and in situ hybridization experiments were performed in combination with immunofluorescent staining on infected cells to localize specific cellular or viral components by electron and light microscopy. The results clearly indicate that none of the viral functions (viral DNA replication, gene expression, splicing or capsid assembly) were present in the pIX-containing c.a. inclusions. However, the POD-associated PML and SP100 proteins were detected in these c.a. inclusions, late in infection. These data indicate that pIX maintains the initial nuclear de-localization of PML protein induced by the early viral E4orf3 protein. Thus pIX contributes to the permanent destabilization of PODs structures during adenovirus infection. The protein pIX is unable to disrupt PODs in a non viral context, but accumulate over PODs and sequester them into c.a. inclusions.

The present invention is based on the discovery that the integrity of POD structures, and thus the cellular activities dependent on POD structures, can be modulated through the action of adenoviral polypeptide(s) capable of interacting with one or more components of the POD structures, such as the adenoviral polypeptides pIX and E4orf3.

On the one hand, it is postulated that expression of the adenoviral polypeptides altering POD's integrity impairs the POD-dependent cellular activities, such as antiviral response and cell apoptosis. With regard to gene therapy vectors, the present invention is expected to provide a reduction of cell toxicity associated with conventional replication-defective adenoviruses, and hence an increase of the maintenance of the therapeutic vector and transgene expression in the treated host cell. In this respect, the present invention provides methods and vectors to reduce or inhibit one or more cellular activitie(s) dependent on said POD nuclear structures, especially for use in the treatment or prevention of disorders in which one or more abnormal POD-dependent cellular activities occur and need to be normalized, such as in chronic disorders and organ degeneration.

On the other hand, it is postulated that enhancement of cell apoptosis may be achieved by suppressing expression of the adenoviral polypeptides altering PODs' integrity. In this respect, the present invention provides methods and vectors for use in the treatment of disorders such as cancers and hyperproliferative disorders where there is insufficient apoptosis.

### DETAILED DESCRIPTION OF BEST MODE AND SPECIFIC/PREFERRED EMBODIMENTS OF THE INVENTION

The term “modulating” as used herein refers to an increase or to a reduction of the POD-dependent cellular activities. Reduction is expected when the adenoviral molecule is provided to the host cell whereas an increase is expected when the function of the adenoviral molecule is abolished in the host cell.

The term “cellular activities(s) dependent on a POD nuclear structure” as used herein refers to at least one activity exerted by or linked to a POD nuclear structure within a host cell, and especially a virally-infected host cell. Such cellular activity may be exerted either directly (e.g., through the action of one or more POD-associated protein(s), for example those described in Negorev and Maul, Oncogene 20 (2001), 7234-7242) or indirectly (through the action of one or more cellular or viral molecules). Such cellular activities include without limitation, regulation of transcription, remodeling of chromatin structure, cellular growth control, differentiation, antiviral response and apoptosis. The modulation of the cellular antiviral response and/or apoptosis is preferred.

The term “contacting” refers to any methods known to a person skilled in the art that are appropriate to bring into contact the molecule of adenoviral origin with the POD nuclear structure of a host cell the cellular activity/activities of which are aimed to be modulated. This “contacting” may for example refer to the contacting of a POD-containing cell or a cell susceptible to form a POD structure (e.g., upon a viral infection) which contains said molecule of adenoviral origin. This may include the use of cultured cells, fixed cells and the like. Preferably, the term “contacting” encompasses embodiments where the molecule of adenoviral origin is introducted into the host cell as is explained in further detail below.

Furthermore, the term “interacting” is defined as providing an effect on the structure and/or one or more functions of a POD nuclear structure. Methods for assessing POD's function and structure are for example disclosed in U.S. Pat. No. 6,319,663. For example, electron microscopy can be used to visually evaluate the POD morphology (e.g., to determine POD disruption). Alternatively, one may directly evaluate levels of a POD-localized protein, such as PML, in order to determine whether its synthesis is up or down regulated. Detection methods include the use of POD-localized protein-specific antibodies such as in in situ hybridization, immunoprecipitation or immunofluorescence assays or the use of appropriate probes to determine the levels of an mRNA encoding the selected POD-localized protein. An evaluation of POD function can be made by the measurement of POD-linked cellular activities, for example by the measurement of nuclear receptor-mediated transcription, or viral replication in infected cells.

The term “molecule” as used herein refers to either a polypeptide or a nucleic acid sequence encoding a polypeptide. Within the present invention, the terms “nucleic acid sequence” and “polynucleotide” are used interchangeably and define a polymeric form of any length of nucleotides or analogs thereof. The term “polynucleotide” includes any possible nucleic acid, in particular DNA, which can be single or double stranded, linear or circular, natural or synthetic. A polynucleotide may comprise modified nucleotides, such as methylated nucleotides and nucleotide analogs (see, U.S. Pat. Nos. 5,525,711; 4,711,955 or EP-302,175 as examples of modifications). Such a polynucleotide can be obtained from existing nucleic acid sources (e.g., genomic, cDNA) but can also be synthetic (e.g., produced by oligonucleotide synthesis). The sequence of nucleotidos may be interrupted by non-riucleotide elements. A polynucleotide may be further modified after polymerization.

The term “polypeptide” is to be understood as a polymeric form of any length of amino acids or analogs thereof. It can be any translation product of a polynucleotide of whatever size and includes peptides but more typically proteins. It is preferably an adenoviral polypeptide encoded by an adenoviral genome. In the context of the present invention, the adenoviral genome can be derived from any adenovirus. An “adenovirus” is any virus of the family Adenoviridae, and desirably of the genus Mastadenovirus (e.g., mammalian adenoviruses) or Aviadenovirus (e.g., avian adenoviruses). The adenovirus can be of any serotype. Adenoviral stocks that can be employed as a source of adenovirus can be amplified from the adenoviral serotypes 1 through 47, which are currently available from the American Type Culture Collection (ATCC, Rockville, Md.), or from any other serotype of adenovirus available from any other source. For instance, an adenovirus can be of subgroup A (e.g., serotypes 12, 18, and 31), subgroup B (e.g., serotypes 3, 7, 11, 14, 16, 21, 34, and 35), subgroup C (e.g., serotypes 1, 2, 5, and 6), subgroup D (e.g., serotypes 8, 9, 10, 13, 15, 17, 19, 20, 22-30, 32, 33, 36-39, and 42-47), subgroup E (serotype 4), subgroup F (serotypes 40 and 41), or any other adenoviral serotype. Preferably, however, an adenovirus is of serotype 2, 5 or 9.

The adenoviral molecule used in the context of the present invention is capable alone or in combination, directly or by means of other cellular or viral molecules, to interact with the cellular function of a POD nuclear structure, as described above. In the context of the present invention, the term “cellular function” refers to the regulation of any cellular process, in particular including the regulation of transcription, cellular growth control, the control of differentiation, antiviral response, apoptosis arid remodeling of chromatin structure.

Most suitably, the adenoviral molecule is or encodes a native full length adenoviral polypeptide from the initiator codon to the stop codon. However, it is also feasible to employ a mutant provided that the modulating property of one or more POD functions be preserved. The term “mutant” refers to a molecule differing from the native adenoviral molecule which retains essential properties of the native molecule. Generally, mutants can be obtained by deletion, addition and/or substitution of one or more nucleotides or of a fragment of nucleotides of the adenoviral polypeptide encoding sequence at any position of the native sequence. Such modifications can be obtained by standard recombinant techniques (i.e., site-directed mutagenesis, enzyme restriction cutting and relegation, PCR techniques and the like). Advantageously, in the context of the present invention, a mutant-encoding sequence shares a high degree of homology with the native sequence, in particular at least 70% identity, more preferably at least 80% and even more preferred at least 90%. Particularly preferred is an absolute identity. By a mutant having an identity of at least 70% with the native adenoviral sequence, it is intended that the mutant sequence includes up to 30 differences per each 100 nucleotides of the native sequence, which can either be silent or can result in a modification of an encoded amino acid residue. As a practical matter, the percentage identity between a mutant and a native sequence can be determined conventionally using known computer programs. A preferred method for determining the best overall match between the mutant and the native sequences, also referred as a global sequence alignment, can be determined using the FASTDB computer program based on the algorithm of Brutlag et at. (Comp. App. Biosci. 6 (1990), 237-245).

The functionality of a mutant can be easily determined by the skilled artisan by comparing the modulating property displayed by the mutant with the modulating property displayed by the native adenoviral polypeptide, either in vitro (by evaluating the POD-associated function(s) in appropriate cultured cells, e.g., IFNg-mediated antiviral response, apoptotic status, observation of PODs morphology), or in vivo (in animal models by evaluating cellular responses to a viral infection such as hepatotoxicity, persistence of a transgene expressed by a recombinant virus), as described hereinafter. In vitro experimental conditions for analyzing POD functions and morphology are provided in examples of the present specification. However, other methods well known by those skilled in the art are also usable in the context of the invention.

According to a first aspect, the present invention provides a method of modulating one or more cellular activitie(s) dependent on a POD nuclear structure in a host cell which comprises introducing in said host cell at least a molecule of adenoviral origin, wherein said molecule of adenoviral origin provides a reduction or an inhibition of one or more cellular activitie(s) dependent on said POD nuclear structure.

The term “host cell” as used herein refers to a single entity, or can be part of a larger collection of cells. Such a larger collection of cells can comprise, for instance, a cell culture (either mixed or pure), a tissue (e.g., epithelial or other tissue), an organ (e.g., heart, lung, liver, urinary bladder, muscle or another organ), an organ system (e.g., circulatory system, respiratory system, gastrointestinal system, urinary system, nervous system, integumentary system or another organ system), or an organism (e.g., a mammal, particularly a human, or the like). Suitable host cells include but are not limited to hematopoietic cells (totipotent, stem cells, leukocytes, lymphocytes, rnoriocytes, macrophages, APC, dendritic cells and the like), pulmonary cells tracheal cells, hepatic cells, epithelial cells, endothelial cells, fibroblasts or muscle cells (cardiac, smooth muscle and skeletal, such as myoblasts, myotubes, myofibers and satellite cells). Preferably, the cells are selected from the group consisting of heart, blood vessel, lung, liver, and muscle cells. Moreover, according to a specific embodiment, the eukaryotic host cell can be further encapsulated. Cell encapsulation technology has been previously described (Tresco et at., ASAIO J. 38 (1992), 17-23; Aebischer et al., Human Gene Ther. 7 (1996), 851-860). The term “host cell” also encompasses complementing cell lines for adenoviral vector or AAV production, such as 293, PERC-6 or 293 E4 orf6/7 cells. The introduction of the POD-modulating adenoviral molecule in such complementing cell lines is expected to improve adenoviral or AAV vector production by reducing the cellular antiviral or apoptosis activities dependent on PODs.

The term “introducing” as used herein refers to any method known to those skilled in the art to introduce a molecule into a cell in the form of a polypeptide or a nucleic acid, including but not limited to transduction, transfection, microinjection, electroporation, viral infection of host cells, endocytosis, use of transporters (e.g., Ad penton base, HIV TAT protein and the like), fusion with a nuclear localization signal (NLS) and receptor-mediated transduction.

In a first embodiment, the host cell is infected by a virus and the adenoviral molecule provides a reduction or an inhibition of the antiviral cellular activity dependent on said POD nuclear structures. The term “virus” encompasses wild type viruses as well as genetically-engineered viruses of any family. Moreover, the viral infection can result from an opportunist infection or from a deliberately-induced infection (e.g., infection by a gene therapy vector such as adenoviral or MV vectors). Preferably, the host cell-infecting virus is a replication-defective adenoviral vector. The term “adenoviral vector” as used herein encompasses vector DNA (genome) as well as viral particles (virus, virioris).

Replication-defective adenoviral vectors are known in the art and can be defined as being deficient in one or more regions of the adenoviral genome that are essential to the viral replication (e.g., E1, E2 or E4 or combination thereof), and thus unable to propagate in the absence of trans-comptementatiori (e.g., provided by either complementing cells or a helper virus). The replication-defective phenotype is obtained by introducing modifications in the viral genomo to abrogate the function of one or more viral gene(s) essential to the viral replication. Such modification(s) include the deletion, insertion and/or mutation (i.e., substitution) of one or more nucleotide(s) in the coding sequence(s) and/or the regulatory sequence(s). Deletions are preferred in the context of the present invention. In this context, the replication-defective vector preferably lacks at least a functional adenoviral E1 region or is a E1-deleted adenoviral vector. Such E1-deleted adenoviral vectors include those described in U.S. Pat. Nos. 6,063,622; 6,093,567; WO 94/28152; WO 98/55639 and EP-974,668 and, the disclosures of all of these publications are hereby incorporated herein by reference. A preferred E1 deletion covers approximately the nuoleotidos (nt) 459 to 3328 or 459 to 3510, by reference to the sequence of the human adenovirus type 5 (disclosed in the GeneBank under the accession number M 73260 and in Chroboczek et al., Virol. 186 (1992), 280-285).

Furthermore, the adenoviral backbone of the vector may comprise modifications in additional viral region(s). In this regard, the adenoviral vector may also be defectivefor the E2 region (either within the E2A or the E2B region or within both the E2A and the E2B region). An example of an E2 modification is illustrated by the thermosensitive mutation of the DBP (DNA Binding Protein) encoding gene (Erisinger et al., J. Virol. 10 (1972), 328-339). The adenoviral vector may also be deleted of all or part of the E4 region (see, for example, EP-974,668 and WO 00/12741). Additional deletions within the non-essential E3 region may increase the cloning capacity, but it may be advantageous to retain all or part of the E3 sequences coding for the polypeptides (e.g., gp19k) allowing to escape the host immune system (Gooding et al., Critical Review of Immunology 10 (1990), 53-71) or inflammatory reactions (EP 00/440267.3). It is also conceivable to employ a minimal (or gutless) adenoviral vector which lacks all functional genes including early (E1, E2, E3 and E4) and late genes (L1, L2, L3, L4 and L5) with the exception of cis-acting sequences (see for example Kovesdi et al., Current Opinion in Biotechnology 8 (1997), 583-589; Yeh and Perricaudet, FASEB 11 (1997), 615-623; WO 94/12649; WO 94/28152). The replication-deficient adenoviral vector may be readily engineered by one skilled in the art, taking into consideration the required minimum sequences, and is not limited to these exemplary embodiments. In this context, the host cell can be infected by an adenoviral vector lacking E1, or E1 and E2, or E1 and E3, or E1 and E4, or E1 and E2 and E3, or E1 and E2 and E4, or E1 and E3 and E4, or E1 and E2 and E3 and E4.

in a preferred embodiment, the host cell is infected by a replication-defective adenoviral vector deficient for E1 and E4 functions, and optionally for E3 function. As an illustration, a preferred E4 deletion covers approximately the nucleotides from position 32994 to position 34998 and a preferred E3 deletion covers approximately the nucleotides at position 28592 to position 30748, by reference to the sequence of the human adenovirus type 5 (disclosed in the GeneBank under the accession number M 73260 and in Chroboczek et al., Virol. 186 (1992), 280-285).

In one embodiment of the method of the present invention, the replication-defective adenoviral vector further comprises a transgene.

The term “transgene” refers to a nucleic acid which can be of any origin and isolated from a genomic DNA, a cDNA, or any DNA encoding a RNA, such as a genomic RNA, a mRNA, an antisense RNA, a ribosomal RNA, a ribozyme or a transfer RNA. The transgene can also be an oligonucleotide (i.e., a nucleic acid having a short size of, for instance, less than 100 bp). The transgene can be engineered from genomic DNA to remove all or part of one or more intronic sequences (i.e., minigene).

In a preferred embodiment, the transgene in use in the present invention, encodes a gene product of therapeutic interest. A “gene product of therapeutic interest” is one which has a therapeutic or protective activity when administered appropriately to a patient, especially a patient suffering from a disease or illness condition or who should be protected against such a disease or condition. Such a therapeutic or protective activity can be correlated to a beneficial effect on the course of a symptom of said disease or said condition. It is within the reach of the man skilled in the art to select a transgene encoding an appropriate gene product of therapeutic interest, depending on the disease or condition to be treated. In a general manner, his choice may be based on the results previously obtained, so that he can reasonably expect, without undue experimentation, i.e., other than practicing the invention as claimed, to obtain such therapeutic properties.

In the context of the invention, the transgene can be homologous or heterologous to the host cell into which it is introduced. Advantageously, it encodes a polypeptide. In the context of transgenes, the term “polypeptide” is to be understood as any translational product of a polynucleotide whatever its size is, and includes polypeptides having as few as 7 residues (peptides), but more typically proteins. In addition, it may be from any origin (prokaryotes, lower or higher eukaryotes, plant, virus etc). It may be a native polypeptide, a variant, a chimeric polypeptide having no counterpart in nature or fragments thereof. Advantageously, the transgene in use in the present invention encodes at least one polypeptide that can compensate for one or more defective or deficient cellular proteins in an animal or a human organism. A suitable polypeptide may also be immunity conferring and may act as an antigen to provoke a humoral or a cellular response, or both.

Preferred transgenes for use in the method of the present invention include, without limitation, those encoding:

polypeptides involved in the cellular cycle, such as p21, p16, the expression product of the retinoblastoma (Rb) gene, kinase inhibitors (preferably of the cyclin-dependent type), GAX, GAS-1, GAS-3, GAS-6, Gadd45 and cyclin A, B and D;

cytokines (including iriterloukins, in particular IL-2, IL-6, IL-B, IL-12, colony stimulating factors such as GM-CSF, G-CSF, M-CSF), IFNα, IFNβ or IFNγ;

polypeptides capable of decreasing or inhibiting a cellular proliferation, including antibodies or polypeptides inhibiting an oncogen expression product (e.g., ras, map kinase, tyrosine kinase receptors, growth factors), Fas ligand, polypeptides activating the host immune system (MUC-1, early or late antigen(s) of a papilloma virus and the like);

polypeptides capable of inhibiting a bacterial, parasitic or viral infection or its development, such as antigenic determinants, transdominant variants inhibiting the action of a viral native protein by competition (EP-614,980, WO 95/16780), the extracellular domain of the HIV receptor CD4 (Traunecker et al., Nature 331 (1988), 84-86), immunoadhesin (Capon et al., Nature 337 (1989), 525-531; Byrn et at., Nature 344 (1990), 667-670), and antibodies (Buchacher et al., Vaccines 92 (1992), 191-195);

immunostirnulatory polypeptides such as B7.1, B7.2, ICAM and the like;

enzymes, such as urease, renin, thrombin, metalloproteinase, nitric oxide synthases (eNOS and iNOS), SOD, catalase, heme oxygenase, the lipoprotein lipase family;

oxygen radical scavengers;

enzyme inhibitors, such as antithrombin III, ptasminogen activator inhibitor PAI-1, tissue inhibitor of metalloproteinase 1-4;

lysosomal storage enzymes, including glucocerebrosidase (Gaucher's disease; U.S. Pat. Nos. 5,879,680 and 5,236,838), alpha-galactosidase (Fabry disease; U.S. Pat. No. 5,401,650), acid alpha-glucosidase (Pompe's disease; WO 00/12740), alpha n-acetylgalactosaminidase (Schindler disease; U.S. Pat. No. 5,382,524), acid sphingomyelinaSe (Niemann-Pick disease; U.S. Pat. No. 5,686,240) and alpha-iduronidase (WO 93/10244),

a protein that can be employed in the treatment of an inherited disease, e.g., CFTR (for the treatment of cystic fibrosis), dystrophin or minidystrophin (for the treatment of muscular dystrophies), alpha-antitrypsin (for the treatment of emphysema), insulin (in the context of diabetes) and hemophilic factors (for the treatment of hemophilias and blood disorders), such as Factor V11a (U.S. Pat. No. 4,784,950), Factor VIII (U.S. Pat. No. 4,965,199) or a derivative thereof (U.S. Pat. No. 4,868,112 having the B domain deleted) and Factor IX (U.S. Pat. No. 4,994,371);

angiogenesis inhibitors, such as angiostatin, eridostatin, platelet factor-4;

transcription factors, such as nuclear receptors comprising a DNA binding domain, a ligand binding domain and a domain activating or inhibiting transcription (e.g., fusion products derived from oestrogen, steroid and progesterone receptors);

markers (beta-galactosidase, CAT, luciferase, GFP and the like); and

any polypeptides that are recognized in the art as being useful for the treatment or prevention of a clinical condition.

As mentioned above, the transgene also includes genes encoding antisense sequences, ribozymes or RNA molecules capable of exerting RNA interference (RNAi), each of these molecules being capable of binding and inactivating specific cellular RNA, preferably that of selected positively-acting growth regulatory genes, such as oncogenes and protooncogenes (c-myc, c-fos, c-jun, c-myb, c-ras, Kc and JE).

It is within the scope of the present invention that the transgene may include addition(s), deletion(s) and/or modification(s) of one or more nucleotide(s) with respect to the native sequence.

In one embodiment, the transgene is operably linked to regulatory elements allowing its expression in a host cell. Such regulatory elements include a promoter, and optionally an enhancer that may be obtained from any viral, bacterial or eukaryotic gene (even from the cellular gene from which the transgene originates) and may be constitutive or regulable. Optionally, it can be modified in order to improve its transcriptional activity, delete negative sequences, modify its regulation, introduce appropriate restriction sites etc. Examples of constitutive promoters include, without limitation, the retroviral Rous sarcoma virus (RSV) promoter (optionally with the RSV enhancer), the cytomegalovirus (CMV promoter) (Boshart et al., Cell 41(1985), 521-530), the SV40 promoter, the dihydrofolate reductase promoter, the beta-actin promoter, the phosphoglycero kinase (PGK promoter; Hitzeman et al., Science 219 (1983), 620-625; Adra et al., Gene 60 (1987), 65-74), especially from mouse or human origin. Inducible promoters are regulated by exogenously supplied compounds, and include, without limitation, the zinc-inducible metallothionein (MT) promoter (Mclvor et al., Mol. Cell Biol. 7 (1987), 838-848), the dexamethasone (Dex)-inducible mouse mammary tumor virus (MMTV) promoter, the T7 polymerase promoter system (WO 98/10088), the ecdysone insect promoter (No et al., Proc. Natl. Acad. Sci. USA 93 (1996), 3346-3351), the tetracycline-repressible promoter (Gossen et al., Proc. Natl. Acad. Sci. USA 89 (1992), 5547-5551), the tetracycline-inducible promoter (Kim et al., J, Virol. 69 (1995), 2565-2573), the RU486-inducible promoter (Wang et al., Nat. Biotech. 15 (1997), 239-243 and Wang et al., Gene Ther. 4 (1997), 432-441) and the rapamycin-inducible promoter (Magari et at., J. Clin. Invest. 100 (1997), 2865-2872). The promoter in use in the content of the present invention can also be tissue-specific to drive expression of the transgene in the tissues where therapeutic benefit is desired. Tissue-specific promoters include promoters from SM22 (WO 98/15575; WO 97135974), Desmin (WO 96/26284), alpha-i antitrypsin (Ciliberto et al., Cell 41 (1985), 531-540), CFTR, surfactant, immunoglobulin genes and SRalpha. Alternatively, one may employ a promoter capable of being activated in proliferative cells isolated from genes overexpressed in tumoral cells, such as the promoters of the MUC-1 gene overexpressed in breast and prostate cancers (Chen et al., J. Clin. Invest. 96 (1995), 2775-2782), of the CEA (Carcinoma Embryonic Antigen)-encoding gene overexpressed in colon cancers (Schrewe et al., Mol. Cell. Biol. 10 (1990), 2738-2748), of the ERB-2 encoding gene overexpressed in breast and pancreas cancers (Harris et al., Gene Therapy 1 (1994), 170-175) and of the alpha-foetoprotein-encoding gene overexpressed in liver cancers (Kanai et al., Cancer Res. 57 (1997), 461-465).

Those skilled in the art will appreciate that the present invention may further use additional control sequences for proper initiation, regulation and/or termination of transcription and translation of the transgene(s) into the host cell or organism. Such control sequences include but are not limited to non-coding exons, introns, targeting sequences, transport sequences, secretion signal sequences, nuclear localization signal sequences, IRES, polyA transcription termination sequences, tripartite leader sequences, sequences involved in replication or integration. Said control sequences have been reported in the literature and can be readily obtained by those skilled in the art.

The adenoviral vector may comprise one or more transgene(s). In this regard, the different transgenes may be controlled by the same (polycistronic) or by separate regulatory elements which can be inserted into various sites within the vector in the same or opposite directions.

In one embodiment of the method of the present invention, the molecule of adenoviral origin is a polypeptide capable of providing a reduction or an inhibition of one or more cellular activities dependent on the POD nuclear structures. In another, and preferred, embodiment of the method of the present invention, the molecule of adenoviral origin is a nucleic acid sequence encoding a polypeptide capable of providing a reduction or an inhibition of one or more cellular activities dependent on the POD nuclear structure.

In a preferred embodiment, the polypeptide of adenoviral origin providing a reduction or an inhibition of one or more cellular activitie(s) dependent on said POD nuclear structures, is selected from the group consisting of pIX and E4orf3, taken individually or in combination. One may therefore consider to provide or express in the host cell either pIX or E4orf3 or both pIX and E4orf3 in order to reduce or inhibit one or more cellular activities dependent on POD nuclear structures. More specifically, said polypeptide of adenoviral origin may be obtained or derived from adenovirus serotype 2 or 5. Based upon the experimental observations described hereinafter, pIX may interfere particularly with the POD-dependent functions through the sequestration of PODs, whereas E4orf3 may act particularly through the disorganization of the POD nuclear structures. As a result, the expression of one or both adenoviral polypeptides in a host cell may inhibit or reduce the POD-dependent functions in this host cell. Both adenoviral sequences can be cloned by applying standard molecular biology from an adenovirus genome as those cited above (and preferably from Ad2 or AdS). Although these adenoviral genes may vary between the different adenovirus strains, they can be identified on the basis of nucleotide and/or amino acid sequences available from different sources (e.g., GeneBank) or by homology with the corresponding well characterized AdS sequences (disclosed in GeneBank under accession number M73260 or in Chroboczek et al., Virol. 186 (1992), 280-285). As an indication, the pIX gene is located at the left hand of the adenoviral genome (between nucleotides 3609 to 4031 in Ad5) whereas the E4orf3-encoding gene is located at the right hand of the adenoviral genome (between nucleotides 34706 (ATG codon) to 34358 (STOP codon) in Ad2).

As mentioned above, it is feasible to employ a mutant of the adenoviral polypeptide(s) to reduce or inhibit one or more cellular activities dependent on POD nuclear structures. In terms of amino acid residues, the mutant polypeptide preferably comprises conservative amino acid substitutions, i.e., such that a given amino acid is substituted by another amino acid of similar size, charge density, hydrophobicity/hydrophilicity, and/or configuration (e.g., Val for Phe). Preferably, a mutant used in the present invention exhibits POD-modulating properties to approximately the same extent as or to a greater extent than the corresponding native adenoviral polypeptide. As described above, the capacity of pIX to sequester POD nuclear structures is mediated by its coiled-coil leucine-rich domain located in the C-terminal portion of pIX. Therefore, one may envisage to use pIX mutants containing modifications in the N-terminal or central portion of the protein, which preserve POD-modulating functions. However, when pIX is expressed by the infecting recombinant adenoviral vector, it is preferred to employ a nucleic acid sequence encoding the wild-type pIX protein, in order to preserve the capsidic and POD-modulating functions of pIX.

More suitably, the native pIX sequences present in the replication-defective adenoviral vector at the 3′ border of the E1 deletion are retained (they are controlled by the native pIX promoter that is non-functional in the absence of replication in the host cell) and the replication-defective adenoviral vector comprises additional pIX encoding sequences placed under the control of an heterologous promoter allowing expression in the host cell.

In a preferred embodiment, the nucleic acid sequence encoding a polypeptide of adenoviral origin having POD-modulating properties is placed under the control of appropriate transcriptional and translational regulatory elements allowing expression in the host cell. For this purpose, the nucleic acid sequence can be placed under the control of a heterologous (non native) promoter. Such a heterologous promoter may be selected from the group consisting of constitutive, inducible, tumor-specific and tissue-specific promoters, such as those defined above in connection with the regulatory elements controlling transgene expression. Preferably, the promoter governing expression of the adenoviral polypepticie is the CMV promoter. Moreover, the regulatory elements may further comprise additional elements, such as one or more enhancers, exonhintron sequences, nuclear localization signal sequences, polyA transcription termination sequences. Said elements have been reported in the literature and can be readily obtained by those skilled in the art.

As a first alternative, the nucleic acid sequence encoding a POD-modulating polypeptide of adenoviral origin is carried by the replication-defective adenoviral vector as defined above. As mentioned above, the method of the present invention preferably uses a recombinant adenoviral vector deleted of both E1 and E4 regions, and optionally of the E3 region. Although the nucleic acid sequence encoding the polypeptide of adenoviral origin can be inserted at any location in said replication-defective adenoviral vector, it is advantageously inserted in replacement of the deleted E4 or E3 region and the transgene is inserted in replacement of the deleted E1 region. Preferably, the polypeptide of adenoviral origin and the transgene are placed under the control of independent transcriptional and translational regulatory elements. It is preferred that the nucleic acid sequence encoding a polypeptide of adenoviral origin and the transgene are transcribed in antisense orientation to each other. As mentioned above, the replication-defective adenoviral vector may retain the native pIX sequence (equipped with the pIX promoter) at its native location (downstream of the E1 region) which are not expressed due to the absence of replication, but may further comprise the nucleic acid sequence encoding pIX under the control of a heterologous promoter and located in said adenoviral vector at a position different from its native location (e.g., in replacement of the deleted E4 or ES region).

According to a second alternative, the nucleic acid sequence encoding a polypeptide of adenoviral origin is carried by a vector different from said replication-defective adenoviral vector, In the context of the present invention, the vector can be a plasmid or a viral vector. The term “plasmid” denotes an extra chromosomal circular DNA capable of autonomous replication in a given cell. The range of suitable plasinids is very large. Preferably, the plasmid is designed for amplification in bacteria and for expression in an eukaryotic target cell. Such plasmids can be purchased from a variety of manufacturers. Suitable plasmids include but are not limited to those derived from pBR322 (Gibco BRL), pUC (Gibco BRL), pBluescript (Stratagene), pREP4, pCEP4 (lnvitrogene), pCI (Promega) and p Poly (Lathe et al., Gene 57 (1987), 193-201). It can also be engineered by standard molecular biology techniques (Sambrook et al., Laboratory Manual, Cold Spring Harbor Laboratory Press, Cold Spring Harbor, N.Y. (2001)). It may also comprise a selection gene in order to select or to identify the transfected cells (e.g., by complementation of a cell autotrophy or by antibiotic resistance), stabilizing elements (e.g., cer sequence; Summers and Sherrat, Cell 36 (1984), 1097-1103) or integrative elements (e.g., LTR viral sequences and transposons). A viral vector may be derived from any virus, especially from herpes viruses, cytomegaloviruses, foamy viruses, lentiviruses, Semliki forrest virus, AAV (adeno-associated virus), poxviruses, adenoviruses and retroviruses. Such viral vectors are well known in the art. “Derived” means genetically engineered starting from the native viral genome by introducing one or more modifications, such as deletion(s), addition(s) and/or substitution(s) of one or several rtucleotide(s) in a coding or a non-coding portion of the viral genome.

Moreover, the vector containing the nucleic acid sequence encoding the POD-modulating adenoviral polypepticie in use in the method of the invention can further comprise a transgene operably linked to appropriate transcriptional arid/or translational regulatory elements allowing its expression in a host cell. With respect to the nature of the transgene and the regulatory elements, the same applies as already set forth previously.

With respect to the two-vector embodiment (second alternative), the method of the present invention comprises introducing in said host cell simultaneously or sequentially (i) said replication-defective adenoviral vector and (ii) said vector comprising said nucleic acid sequence encoding said polypeptide of adenoviral origin. “Sequentially” means that at least the replication-defective adenoviral vector and the vector encoding said polypeptide of adenoviral origin are introduced in the host cell or organism one after the other. If the two vectors are sequentially administered, preferably the vector encoding said polypeptide of adenoviral origin is administered subsequently to the replication-defective adenoviral vector. Sequential administration of the second vector, such as the vector encoding said polypeptide of adenoviral origin, can be immediate or delayed and can be done by the same route or a different route of administration. If sequential administration of the second vector is delayed, the delay can be a matter of minutes, hours, days, weeks, months or even longer.

In the context of the method of the present invention, the vector encoding the POD-modulating adenoviral polypeptide may be complexed with various compounds that can improve vector delivery efficiency or stability. Such compounds include but are not limited to lipids, polymers, peptides, condensing agents (spermine, sperrnidine, histones, peptides) and their derivatives. These compounds are widely described in the scientific literature accessible to the man skilled in the art.

In this respect, preferred lipids are cationic lipids which have a high affinity for nucleic acids (e.g., the vector of the present invention) and which interact with cell membranes (Feigner et al., Nature 337 (1989), 387-388). As a result, they are capable of complexing the nucleic acid, thus generating a compact particle capable of entering the cells. Cationic lipids or mixtures of cationic lipids which may be used in the present invention include Lipofectin™, DOTMA: N-[1-(2,3-dioleyloxyl)propy]-N,N,N-trimethylammonium (Feigner, Proc. Natl. Acad. Sci. USA 84 (1987), 7413-7417), DOGS: dioctadecylamidoglycylspermine or Transfectam™ (Behr, Proc. Natl. Acad. Sci. USA 86 (1989), 6982-6986), DMR1E: 1,2-dimiristyloxypropyl-3-dimethyl-hydroxyethylammonium and DORIE: 1,2-diooleyloxypropyl-3-dimethyl-hydroxyethylammonium (Feigner, Methods 5 (1993), 67-75), DC-CHOL: 3 [N-(N′,N′-dimethyiamioethane)-carbamoyl]Cholesterol (Gao, BBRC 179 (1991), 280-285), DOTAP (McLachlan, Gene Therapy 2 (1995), 674-622), Lipofectamine™ spermine-and spermidine-cholesterol, Lipofectace™ (for a review, see, for example, Legendre, Medecine/Scieflce 12 (1996), 1334-1341 or Gao, Gene Therapy 2 (1995), 710-722) and the oationic lipids disclosed in patent applications WO 98/34910, WO 98/14439, WO 97/19675, WO 97/37966 and their isomers. Nevertheless, this list is not exhaustive and other cationic lipids well known in the art can be used in connection with the present invention as well.

Cationic polymers or mixtures of cationic polymers which may be used in the present invention include chitosan (WO 98/17693), poly(aminoacids) such as polylysine (U.S. Pat. No. 5,595,897 or FR 2,719,316); polyquaternary compounds; protamine; polyimines; polyethylene imine or polypropylene imine (WO 96/02655); polyvinylamines; polycationic polymer derivatized with DEAEI such as DEAE dextran (Lopata et al., Nucleic Acid Res. 12 (1984), 5707-5717); polyvinyipyridine; polymethacrylates polyacrylates; polyoxethanes; polythiodiethylaminomethaylethylene (P(TDAE)); polyhistidine; polyornithine; poly-p-aminostyrefle polyoxethanes; co-polymethacrylates (e.g., copolymer of HPMA; N-(2-hydroxypropyl)-methacrylamide); the compound disclosed in U.S. Pat. No. 3,910,862, polyvinytpyrrolid complexes of DEAE with methacryiato, dextran, acrylamide, polyimines, albumin, onedimethylaminomethylmethacrylates and polyvinylpyrrolidone-methylacrylaminopropyltrimethyl ammonium chlorides; polyamidoamifle (Haensier and Szoka, Bioconjugate Chem. 4, (1993), 372-379); telomeric compounds (patent application filing number EP-98 401 471.2); dendritic polymers (WO 95/24221). Nevertheless, this list is not exhaustive and other cationic polymers well known in the art can be used in the composition according to the invention as well.

Colipids may be optionally included in order to facilitate entry of the vector into the cell. Such colipids can be neutral or zwitterionio lipids. Representative examples include phosphatidylethanolamine (PE), phosphatidylcholine, phosphocholine, dioleylphosphatidylethanolamine (DOPE), sphingomyelin, ceramide or cerebroside and any of their derivatives.

The present invention also encompasses the use of replication-defective adenoviral vectors or particles that have been modified to allow preferential targeting of a particular target cell. A characteristic feature of targeted vectors/particles of the invention (whereby said vectors can be of both viral and non-viral origin, such as polymer- and lipid-complexed vectors) is the presence at their surface of a targeting moiety capable of recognizing and binding to a cellular and surface-exposed component. Such targeting moieties include without limitation chemical conjugates, lipids, glycolipids, hormones, sugars, polymers (e.g., PEG, polylysine, PEI and the like), peptides, polypeptides (for example JTS1 as described in WO 94/40958), oligonucleotides, vitamins, antigens, lectins, antibodies and fragments thereof. They are preferably capable of recognizing and binding to cell-specific markers, tissue-specific markers, cellular receptors, viral antigens, antigenic epitopes or tumor-associated markers. The specificity of infection of adenoviruses is determined by the attachment to cellular receptors present at the surface of permissive cells. In this regard, the fiber and penton present at the surface of the adenoviral capsid play a critical role in cellular attachment (Defer et al., J. Virol. 64 (1990), 3661-3673). Thus, cell targeting of adenoviruses can be carried out by genetic modification of the viral gene encoding fiber and/or periton, to generate modified fiber and/or penton capable of specific interaction with unique cell surface polypeptides. Examples of such modifications are described in the literature (for example in Wickam et al., J. Virol. 71 (1997), 8221-8229; Arnberg et al., Virol. 227 (1997), 239-244; Michael et al., Gene Therapy 2 (1995), 660-668; WO 94/10323). As an illustrative example, inserting a sequence coding for EGF within the sequence encoding the adenoviral fiber will allow to target EGF receptor expressing cells. Other methods for achieving cell-specific targeting involve the chemical conjugation of targeting moieties at the surface of the replication-defective adenoviral vector.

In a further embodiment of the method of the present invention, the molecule of adenoviral origin provides a reduction or an inhibition of apoptosis in said host cell. Such a reduction or inhibition can be evaluated by comparing the apoptotic status of the host cell, tissue or organism in the presence of the molecule used according to the invention compared to its absence or the absence of its expression. As a result, the host cell, tissue or organism comprising said molecule is less prone to apoptosis (cell death) or is recovering more rapidly or more efficiently than a host cell, tissue or organism not containing or not expressing said molecule. Such a reduction of cell apoptosis can be determined by quantitative and qualitative methods for apoptosis detection and cellular cycle characterization, including Tryptan blue, DAPI, TUNEL, co-focal microscopy, FAGS and ultrastructural analysis. For example, a reduction of apoptosis can be correlated to a reduction of the concentration of one or several markers that are produced in the course of the apoptosis (reduction of the apoptosis associated markers by a factor of at least 2 to 10). Apoptosis-induced morphological changes include the reduction of condensation of chromatin, DNA cleavage, disassembly of nuclear scaffold proteins, formation of apoptotic bodies and/or nuclear fragmentation.

In another embodiment of the method of the present invention, the molecule of adenoviral origin provides a reduction or an inhibition of the toxicity induced by a gene therapy vector (e.g., said replication-defective adenoviral vector) in said host cell and/or an enhancement of the persistence of transgene expression in said host cell. By way of illustration, a reduction of toxicity can be correlated for example to a reduction of the inflammation status in the host organism (which can be evaluated by observation of cell morphology especially at close proximity to the injected site) and/or a reduction of cell infiltration in the expressing tissues (especially 004+ and CD8+ cells, i.e., by immunohistology), and/or a reduction of necrosis or tissue degeneration and/or a reduction of cytokine production following administration of the replication-defective adenoviral vector (such as TNF (Tumor Necrosis factor) alpha, IFN (interferon) gamma, IL (interleukin)-6 and IL-12) and/or a reduction of hepatotoxicity (decrease of transaminases), and/or an improvement of survival of animals mimicking a toxic reaction (an increase of the survival rate by a factor of at least 2 over a period of time of at least 3 days could be interpreted as an improvement of a toxic status). Transgene expression can be determined by evaluating the level of the gene product over a period of time, either in vitro (e.g., in cultured cells) or in vivo (e.g., in animal models), by standard methods such as flow cytofluorimetry, ELISA, immunofluorescence, Western blotting, biological activity measurement and the like. The improvement of gene expression compared to a control not containing or not expressing the adenoviral molecule can be seen in terms of the amount of gene product or in terms of the persistence of the expression (stability over a longer period of time).

The present invention also provides a recombinant adenoviral vector deleted of the E1 and E4 regions, and optionally of the E3 region, comprising at least (i) a transgene and (ii) a nucleic acid sequence encoding a functional adenoviral pIX protein, wherein said nucleic acid sequence encoding the functional adenoviral pIX protein is placed under the control of a heterologous promoter and located in said adenoviral vector in a position different from its native location.

The term “adenoviral vector” is described above in connection with the method of the present invention. “Recombinant” refers to the presence of a transgene the expression of which is desirably beneficial, e.g., prophylactically or therapeutically, to the cell or to a tissue or organism of which the host cell is a part. The term “functional” as used herein means that the pIX protein is able to exert its function (e.g., modulation of one or more POD-dependent cellular activities) in the absence of viral replication (in a host cell). Preferably, the nucleic acid sequence encoding the adenoviral pIX protein is located in replacement of the deleted E4 region or in replacement of the deleted E3 region in the recombinant adenoviral vector. In this context, the recombinant adenoviral vector of the invention may retain the native pIX sequences equipped with the pIX promoter present at the 3′ border of the E1 deletion but which are not functional in the host cell in the absence of viral replication, but further contains a nucleic acid sequence encoding pIX protein under the control of a heterologous promoter (non-pIX gene promoter) to drive expression of a functional pIX gene product in the host cell. As mentioned above, the nucleic acid sequence can encode a wild-type or a mutant pIX gene product, with a special preference for a wild-type pIX. Advantageously, the recombinant adenoviral vector of the present invention can further comprise a nucleic acid sequence encoding an adenoviral E4orf3 protein placed under the control of a heterologous promoter, while lacking the other E4 genes. The E4orf3-encoding gene can be inserted into any location of the adenoviral genome (e.g., into the deleted E4 or E3 region as an expressing cassette together with the pIX gene) and can be controlled by the same or separate transcriptional and translational regulatory elements as the pIX under the control of a heterologous promoter. When the use of a polycistronic expression cassette is considered for the expression of both pIX and E4orf3 sequences, the translation of the second cistron can be reinitiated by means of an IRES. When the use of two expression cassettes is considered, they can be positioned in sense (same transcriptional direction) or antiserise (opposed transcriptional direction) orientation.

The range of suitable heterologous promoters for controlling the expression of either pIX or both pIX and E4orf3 is very large and within the reach of the skilled artisan. The promoter is preferably selected from the group consisting of constitutive, inducible, tumor-specific and tissue-specific promoters. Such promoters are illustrated above in connection with the method of the present invention.

As mentioned before, the term “adenoviral vector” also encompasses viral particles comprising such a vector. Viral particles may be prepared and propagated according to any conventional technique in the field of the art (e.g., as described in Graham and Prevect, Methods in Molecular Biology, Vol. 7, Gene Transfer and Expression Protocols (1991); Murray, The Human Press mc, Clinton, N.J. or in WO 96/17070) using a complementation cell line or a helper virus, which supplies in trans the viral genes for which the adenoviral vector of the invention is defective (at least the E1 functions). When the recombinant adenoviral vector comprises an E4orf3-expressing nucleic acid sequence, it is optional to provide trans-complementation of E4, since the expression of E4orf3 can be sufficient to provide the E4 functions required for DNA replication and late protein synthesis, as reported in U.S. Pat. No. 5,670,488. The cell lines 293 (Graham et al., J. Gen. Virol. 36 (1977), 59-72) and PERC6 (Fallaux et al., Human Gene Therapy 9 (1998), 1909-1917) are commonly used to complement the E1 function. Other cell lines have been engineered to complement doubly defective vectors (Yeh et al., J. Virol. 70 (1996), 559-565; Krougliak and Graham, Human Gene Ther. 6 (1995), 1575-1586; Wang et al., Gene Ther. 2 (1995), 775-783; Lusky et al., J. Virol. 72 (1998), 2022-2033; EP 919627 and WO 97/04119). The adenoviral particles can be recovered from the culture supernatant but also from the cells after lysis and optionally can be further purified according to standard techniques (e.g., chromatography, ultracentrifugation, as described in WO 96/27677, WO 98/00524 WO 98/26048 and WO 00/50573). Moreover, the recombinant adenoviral vector of the invention can be targeted to a particular host cell, as described above.

The present invention also provides a composition comprising the recombinant adenoviral vector of the present invention or the molecule of adenoviral origin in use in the method of the invention, and a pharmaceutically acceptable vehicle. The composition according to the invention may be manufactured in a conventional manner for a variety of modes of administration including systemic, topical and localized administration (e.g., topical, aerosol, instillation, oral administration). For systemic administration, injection is preferred, e.g., subcutaneous, intradermal, intramuscular, intravenous, intraperitoneal, intrathecal, intracardiac (such as transendocardial and pericardial), intratumoral, intravaginal, intrapulmonary, intranasal, intratracheal, intravascular, intraarterial, intracoronary or intracerebroventricular injection. Intramuscular or intravenous injection constitutes the preferred mode of administration. The administration may take place in a single dose or in a dose repeated one or several times after a certain time interval. The appropriate administration route and dosage may vary in accordance with various parameters, as for example, the condition or disease to be treated, the stage to which it has progressed, the need for prevention or therapy and the therapeutic transgene to be transferred. As an indication, a composition may be formulated in the form of doses of between 10⁴ and 10¹⁴ iu (infectious units), advantageously between 10⁵ and 10¹³ iu and preferably between 10⁶ and 10¹² iu. The titer may be determined by conventional techniques. The composition of the invention can be provided in various forms, e.g., in a solid (e.g., powder, lyophilized form), or a liquid (e.g., aqueous) form.

Moreover, the composition of the present invention can further comprise a pharmaceutically acceptable carrier for delivering said recombinant adenoviral vector or said molecule into a human or animal body. The carrier is preferably a pharmaceutically suitable injectable carrier or diluent which is non-toxic to a human or animal organism at the dosage and concentration employed (for example, see Remington's Pharmaceutical Sciences, 16^{th} Ed., Mack Publishing Co (1980)). It is preferably isotonic, hypotonic or weakly hypertonic and has a relatively low ionic strength, such as provided by a sucrose solution. Furthermore, it may contain any relevant solvents, aqueous or partly aqueous liquid carriers comprising sterile, pyrogen-free water, dispersion media, coatings, and equivalents, or diluents (e.g., Tris-HCl, acetate, phosphate), emulsifiers, solubilizers or adjuvants. The pH of the pharmaceutical preparation is suitably adjusted and buffered in order to be appropriate for use in humans or animals. Representative examples of carriers or diluents for an injectable composition include water, isotonic saline solutions which are preferably buffered at a physiological pH (such as phosphate buffered saline, Tris buffered saline, mannitol, dextrose, glycerol containing or not polypeptides or proteins such as human serum albumin). Illustrative examples of such diluents include a sucrose-containing buffer (e.g., 1M saccharose, 150 mM NaCl, 1 mM MgCl₂, 54 mg/l Tween 80, 10 mM Tris pH 8.5) and a mannitol-containing buffer (e.g., 10 mg/ml mannitol, 1 mg/ml HSA, 20 mM Tris pH 7.2 and 150 mM NaCl).

In addition, the composition according to the present invention may include one or more stabilizing substance(s), such as lipids (e.g., oationic lipids, liposomes, lipids as described in WO 98/44143; Feigner et al., Proc. West. Pharmacol. Soc. 32 (1087), 115-121; Hodgson and Solaiman, Nature Biotechnology 14 (1996), 339-342; Remy et al., Bioconjugate Chemistry 5 (1994), 647-654), nuclease inhibitors, hydrogel, hyaluronidase (WO 98/53853), collagenase, polymers, chelating agents (EP 890362), in order to prevent its degradation within the animal/human body and/or to improve delivery into the host cell. Such substances may be used alone or in combination (e.g., cationic and neutral lipids). It may also comprise substances susceptible to facilitate gene transfer for special applications, such as a gel complex of polylysine and lactose facilitating delivery by the intraarterial route (Midoux et al., Nucleic Acid Res. 21(1993), 871-878) or poloxamer 407 (Pastore, Circulation 00 (1994), 1-517). It has also be shown that adenovirus proteins are capable of destabilizing endosomes and enhancing the uptake of DNA into cells. The mixture of adenoviruses to solutions containing a lipid-complexed plasmid vector or the binding of DNA to polylysine covalently attached to adenoviruses using protein cross-linking agents may substantially improve the uptake and expression of the vector (Curiel et al., Am. J, Respir. Cell. Mol. Biol. 6 (1992), 247-252).

The composition of the present invention is particularly intended for the preventive or curative treatment of chronic disorders, conditions or diseases, and especially genetic diseases (e.g., muscular myopathies, hemophilias, cystic fibrosis, diabetes-associated diseases, Fabry disease, Gaucher disease, lysosomal storage diseases, anemias), chronic viral infections (e.g., hepatitis B and C, AIDS), diseases associated with blood vessels, and/or the cardiovascular system (e.g., ischemic diseases, artheriosclerosis, hypertension, atherogenesis, connective tissue disorders, such as rheumatoid arthritis, ocular angiogonic diseases such as macular degeneration, corneal graft rejection, neovascular glaucoma, myocardial infarcts, cerebral vascular diseases), hepatic-associated diseases (e.g., hepatic failure, hepatitis cirrhosis, alcoholic liver diseases, chemotherapy-induced toxicity), immune disorders (e.g., chronic inflammation, autoimmunity and graft rejection), neurodegenerative diseases (e.g., Parkinson disease, sclerosis).

The present invention also provides the use of the recombinant adenoviral vector of the invention, or of the molecule of adenoviral origin in use in the method of the invention to provide a reduction or an inhibition of one or more cellular activities dependent on a POD nuclear structure. In one embodiment, such a use refers to a reduction or inhibition of the antiviral cellular activity dependent on a POD nuclear structure in the host cell when infected by a virus (e.g., a gene therapy vector, and especially a replication-defective adenoviral vector). In another embodiment, said use refers to a reduction or an inhibition of apoptosis in said host cell, especially when said host cell is infected by a virus (e.g., a gene therapy vector such as a replication-detective adenoviral vector). In this context, said virus and said molecule are prepared as described in connection with the method according to the present invention. In a preferred embodiment, the use of the invention refers to a reduction or an inhibition of the toxicity induced by a replication-defective adenoviral vector in said host cell and/or an enhancement of the persistence of transgene expression in said host cell. The administration of conventional gene-therapy vectors may be associated with acute inflammation, toxicity and/or cell death (apoptosis) in the treated organism, which may result in the elimination of the infected cells and rapid loss of transgene expression. The adenoviral vector or the molecule used in the method of the invention may at least partially protect from such apoptotic status and/or toxicity and, thus, may allow a prolonged transgene expression.

The present invention also provides the use of the recombinant adenoviral vector according to the invention, or the molecule as described in connection with the method according to the invention, for the preparation of a medicament intended for gene transfer, preferably into a human or animal body. Within the scope of the present invention, “gene transfer” has to be understood as a method for introducing a transgene into a cell. Thus, it also includes immunotherapy that may comprise the introduction of a potentially antigenic epitope into a cell to induce an immune response which can be cellular or humoral or both.

For this purpose, the recombinant adenoviral vector, or the molecule of adenoviral origin may be delivered in viva to the human or animal organism by specific delivery means adapted to the pathology to be treated, For example, a balloon catheter or a stent coated with the recombinant adenoviral vector or the vector or replication-defective adenoviral vector encoding the POD-modulating adenoviral polypeptide may be employed to efficiently reach the cardiovascular system (as described in Riessen et al., Hum Gene Ther. 4 (1993), 749-758; Feldman and Steg, Medecine/Science 12 (1996), 47-55). It is also possible to deliver these therapeutic agents by direct administration, e.g., intravenously, in an accessible tumor, in the kings by aerosolization and the like. Alternatively, one may employ eukaryotic host cells that have been engineered ex vivo to contain the recombinant adenoviral vector of the invention or the replication-defective adenoviral vector or the vector encoding the POD-modulating adenoviral polypeptide in use in the method of the invention. Methods for introducing such elements into a eukaryotic cell are well known to those skilled in the art and include microinjection of minute amounts of DNA into the nucleus of a cell (Capechi et al., Cell 22 (1980), 479-488), transfection with CaPO₄ (Chen and Okayama, Mol. Cell Biol. 7 (1987), 2745-2752), electroporation (Chu et al, Nucleic Acid Res. 15 (1987). 1311-1326), lipofection/liposome fusion (Feigner et al., Proc. Natl. Acad. Sci. USA 84 (1987), 7413-7417) and particle bombardment (Yang et al., Proc. Natl. Acad. Sci. USA 87 (1990), 9568-9572), The graft of engineered cells is also possible in the context of the present invention (Lynch et al., Proc. Natl, Acad. Sd. USA 89 (1992), 1138-1142).

The present invention also relates to a method for the treatment of a human or animal organism, comprising administering to said organism a therapeutically effective amount of a recombinant adenoviral vector of the invention, or of the molecule as described in connection with the method according to the invention.

A “therapeutically effective amount” is a dose sufficient for the alleviation of one or more symptoms normally associated with the disease or condition desired to be treated. When prophylactic use is concerned, this term means a dose sufficient to prevent or to delay the establishment of a disease or condition.

The method of the present invention can be used for preventive purposes and for therapeutic applications relative to the diseases or conditions listed above. The present method is particularly useful to prevent or reduce an apoptotic and/or toxic response following administration of a conventional gene-therapy vector. It is to be understood that the present method can be carried out by any of a variety of approaches, for example by direct administration in vivo or by the ex vivo approach.

In a second aspect of the present invention, the present invention also provides a replication-competent adenoviral vector, wherein the native adenovirus pIX and/or the E4orf3 gene is nonfunctional or deleted. In a preferred embodiment, both native adenovirus pIX and E4orf3 genes are nonfunctional or deleted. This adenoviral vector is preferentially meant for use in cancer therapy.

It should be stressed that prior art replication-competent adenoviral vectors retain a functional pIX gene and/or a functional E4orf3 able to reduce or inhibit the cellular activitie(s) dependent on POD nuclear structures including antiviral host response and/or apoptosis, thus reducing the capability of the replication-competent adenoviral vector to destroy these structures. On this basis, the present invention proposes to delete or mutate either pIX or E4orf3 or both pIX and E4orf3 adenoviral genes in order to abrogate their respective POD-associated functions with the purpose of enhancing cell destruction. Preferably, the native adenoviral pIX and/or E4orf3 genes are mutated to prevent its (their) expression, for example by introducing a STOP codon into their respective coding sequences. But it is also conceivable to introduce one or more mutations that exclusively abolish the POD-modulating functions of these polypeptides. For example, with respect to pIX, suitable pIX mutants are those that are defective in the POD-modulating function but does not prevent incorporation in the viral capsid. Such pIX mutants are mutated in the C-terminal portion of the pIX protein, and especially in the leucine rich coiled-coil domain, In this regard, the leucine repeat can be disrupted by disturbing the correct alignment of the apolar residues at one or more location(s) or by disturbing hydrophobic bonding. Suitable pIX mutants include those described in Rosa-Calatrava et al., (J. Virol. 71 (2001), 7131-7141) which include the replacement of the leucine residue at position 114 by praline (LI 14P) or the replacement at the valine residue at position 117 by aspartic acid (VI 17D) or the replacement of both the leucine residue at position 114 by proline and that of the valine residue at position 117 by aspartic acid (L-V).

The term “replication-competent” as used herein refers to an adenoviral vector capable of replicating in a host cell in the absence of any trans-complementation. In the context of the present invention, this term also encompasses replication-selective or conditionally-replicative adenoviral vectors which are engineered to replicate better or selectively in cancer or hyperproliferative host cells. Examples of such replication-competent adenoviral vectors are well known in the art and readily available to those skill in the art (see, for example, Hernandez-Alcoceba et al., Human Gene Ther. 11 (2000), 2009-2024; Nemunaitis et al., Gene Ther. 8 (2001)1746-759; Alemany et al., Nature Biotechnology 18 (2000), 723-727). As before, the term “adenoviral vector” encompasses vector DNA as well as viral particles generated thereof by conventional technologies. Moreover, it also includes “targeted” adenoviral vectors that carry at their surface a targeting moiety capable of recognizing and binding to cell-specific markers, tissue-specific markers, cellular receptors, viral antigens, antigenic epitopes or tumor-associated markers. In this regard, cell targeting of adenoviruses can be carried out by genetic modification of the viral gene encoding the adenoviral polypeptide present on the surface of the virus (e.g., fiber and/or penton) or by chemical coupling, as described further above.

Replication-competent adenoviral vectors according to the invention can be a wild-type adenovirus genome or can be derived therefrom by introducing modifications in the viral genome, e.g., for the purpose of generating a conditionally-replicative adenoviral vector. Such modification(s) include the deletion, insertion and/or mutation of one or more nucleotide(s) in the coding sequences and/or the regulatory sequences. Preferred modifications are those that render said replication-competent adenoviral vector dependent on cellular activities specifically present in a tumor or cancerous cell. In this regard, viral gene(s) that become dispensable in tumor cells, such as the genes responsible for activating the cell cycle through p53 or Rb binding can be completely or partially deleted or mutated. By way of illustration, such conditionally-replicative adenoviral vectors can be engineered by the complete deletion of the adenoviral EIB gene encoding the 55 kDa protein or the complete deletion of the EIB region to abrogate p53 binding. As another example, the complete deletion of the EIA region makes the adenoviral vector dependent on intrinsic or IL-6-induced E1A-like activities. In a second strategy, native viral promoters controlling transcription of the viral genes can be replaced with tumor-specific promoters. By way of illustration, regulation of the E1A and/or the E1B genes can be placed under the control of a tumor-specific promoter such as the PSA, the kallikrein, the probasin or the AFP promoter.

In the context of the present invention, the replication-competent adenoviral vector can be derived from any virus of the family Adenoviridae, and desirably of the genus Mastadenovirus (e.g., mammalian adenoviruses) or Aviadenovirus (e.g., avian adenoviruses). The adenovirus can be of any serotype. Adenoviral stocks that can be employed as a source of adenovirus can be amplified from the adenoviral serotypes 1 through 47, which are currently available from the American Type Culture Collection (ATCC, Rockville, Md.), or from any other serotype of adenovirus available from any other source. For instance, an adenovirus can be of subgroup A (e.g., serotypes 12, 18, and 31), subgroup B (e.g., serotypes 3, 7, 11, 14, 16, 21, 34, and 35), subgroup C (e.g., serotypes 1, 2, 5, and 6), subgroup D (e.g., serotypes 8, 9, 10, 13, 15, 17, 19, 20, 22-30, 32, 33, 36-39, and 42-47), subgroup E (serotype 4), subgroup F (serotypes 40 and 41), or any other adenoviral serotype. Preferably, however, an adenovirus is of serotype 2, 5 or 9.

Advantageously, the replication-competent adenoviral vector of the invention further comprises a transgene placed under the control of transcriptional and/or translational regulatory elements to allow its expression in the host cell. As before, the term “transgene” refers to a nucleic acid which can be of any origin and isolated from a genomic DNA, a cDNA, or any DNA encoding a RNA, such as a genomic RNA, a mRNA, an antisense RNA, a ribosomal RNA, a ribozyme or a transfer RNA. The transgene can also be an oligonucleotide (i.e., a nucleic acid having a short size of less than 100 bp). It can be engineered from genomic DNA to remove all or part of one or more intronic sequences (i.e., minigene). In a preferred embodiment, the transgene in use in this aspect of the present invention encodes a gene product having a therapeutic or protective activity when administered appropriately to a patient, especially a patient suffering from a cancer or hyperproliferative disease. Such a therapeutic or protective activity can be correlated to a beneficial effect on the course of a symptom of said disease or said condition. The transgene can be homologous or heterologous to the host cell into which it is introduced. It is within the reach of the man skilled in the art to select a transgene encoding an appropriate antitumoral gene product. In a general manner, his choice may be based on the results previously obtained, so that he can reasonably expect, without undue experimentation, i.e., other than practicing the invention as claimed, to obtain such therapeutic properties. Advantageously, the transgene encodes a polypeptide (any translational product of a polynucleotide whatever its size) from any origin (prokaryotes, lower or higher eukaryotes, plant, virus etc). It may be a native polypeptide, a variant, a chimeric polypeptide having no counterpart in nature or fragments thereof. Advantageously, the transgene in use in the present invention encodes at least one polypeptide that acts through toxic effects to limit or remove harmful cells from the body.

Preferred transgenes include, without limitation, suicide genes, genes encoding toxins, immunotoxins (Kurachi et al., Biochemistry 24 (1985), 5494-5499), lytic polypeptides, cytotoxic polypeptides, apoptosis inducers (such as p53, Bas, Bc12, BcIX, Bad and their antagonists) and angiogenic polypeptides (such as members of the family of vascular endothelial growth factors, VEGF; i.e., heparin-binding VEGF GeneBank accession number M32977) transforming growth factor (TGF, and especially TGFα and β), epithelial growth factors (EGF), fibroblast growth factor (FGF and especially FGFα and β), tumor necrosis factors (TNF, especially TNFα and β), CCN (including CTGF, Cyr61, Nov, E1m-1, Cop-i and Wisp-3), scatter factor/hepatocyte growth factor (SH/HGF), angiogenin, angiopoietin (especially 1 and 2), angiotensin-2, plasminogen activator (tPA) arid urokinase (uPA).

In a preferred embodiment, the transgene is a suicide gene. In the context of the invention, the term “suicide gene” encompasses any gene whose product is capable of converting an inactive substance (prodrug) into a cytotoxic substance, thereby giving rise to cell death. The gene encoding the thymidine kinase (1K) of HSV-1 constitutes the prototype of the suicide gene family (Caruso et al., Proc. Natl. Acad. Sci. USA 90 (1993), 7024-7028; Culver et al., Science 256 (1992), 1550-1552). TK catalyzes the transformation of nucleoside analogs (prodrug) such as acyclovir or ganciclovir to toxic nucleosides that are incorporated into the neoformed DNA chains, leading to inhibition of cell division. A large number of suicide gene/prodrug combinations are currently available. In the context of the invention of particular interest are rat cytochrome p450 and cyclophosphophamide (Wei et al., Human Gene Ther. 5 (1994), 969-978), Escherichia coil (E. coli) purine nucleoside phosphorylase and 6-methylpurine deoxyribonucleoside (Sorscher et al., Gene Therapy 1 (1994), 223-238), E. coli guanine phosphtioribosyl transferase and 6-thioxanthine (Mzoz et al., Human Gene Ther. 4 (1993), 589-595). However, in a more preferred embodiment, the replication competent adenoviral vector of the invention comprises a suicide gene encoding a polypeptide having a cytosine deaminase (CDase) or a uracil phosphoribosyl transferase (UPRTase) activity or both CDase and UPRTase activities, which can be used with the prodrug 5-fluorocytosine(5-FC). The use of a combination of suicide genes, e.g., encoding polypeptides having CDase and UPRTase activities, can also be envisaged in the context of the invention.

CDase and UPRTase activities have been demonstrated in prokaryotes and lower eukaryotes, but are not present in mammals. CDase is normally involved in the pyrimidine metabolic pathway by which exogenous cytosine is transformed into uracil by means of a hydrolytic deamination, whereas UPRTase transforms uracile in UMP. However, CDase also deaminates an analog of cytosine, 5-FC, thereby forming 5-fluorouracil (5-FU), which is highly cytotoxic when it is converted into 5-fluoro-UMP (5-FUMP) by UPRTase activity.

Suitable CDase encoding genes include but are not limited to the Saccharomyces cerevisiae FCY1 gene (Erbs et al., Curr. Genet. 31(1997), 1-6; WO 93/01281) and the E. coil codA gene (EP 402 108). Suitable UPRTase encoding genes include but are not limited to those from E. coli (upp gene; Anderson et al., Eur. J. Biochem. 204 (1992), 51-56), Lactococcus lactis (Martinussen and Hammer, J. Bacteriol. 176 (1994), 6457-6463), Mycobacterium bovis (Kim et al., Biochem. Mol. Biol. Int 41 (1997), 1117-1124), Bacillus subtilis (Martinussen et al., J. Bacteriol. 177 (1995), 271-274) and Saccharomyces cerevisiae (FUR-1 gene; Kern et al., Gene 88 (1990), 149-157). Preferably, the CDase encoding gene is derived from the FCY1 gene and the UPRTase encoding gene is derived from the FUR-1 gene.

The present invention also encompasses the use of mutant suicide genes, modified by the addition, deletion and/or substitution of one or several nucleotides providing that the cytotoxic activity of the gene product be preserved. A certain number of CDase and UPRTase mutants have been reported in the literature. Preferably, the suicide gene in use in the present invention encodes a fusion polypeptide having both the CDase and the UPRTase activity (WO 96/16183). In a particularly preferred embodiment, the fusion polypeptide comprises a mutant of the UPRTase encoded by the FUR-I gene having the first 35 residues deleted (mutant FCU-1 disclosed in WO 99/54481).

The replication-competent adenoviral vector may comprise one or more transgene(s). In this regard, the combination of genes encoding a suicide gene product and a cytokine (such as IL-2, IL-8, IFNγ, GM-CSF) or an immunostimulatory polypeptide (such as B7.1, B7.2, ICAM and the like) may be advantageous in the context of the invention. The different transgenes may be controlled by the same (polycistronic) or by separate regulatory elements which can be inserted into various sites within the vector, in the same direction or in opposite directions.

Preferably, the regulatory elements controlling expression of the transgene in the host cell comprise a tumor-specific promoter. Such promoters are known in the art. Representative examples are described above in connection with the method of the present invention.

The present invention also provides a method for preparing a viral particle comprising:

(i) introducing the replication-competent adenoviral vector of the invention into a permissive cell, to obtain a transfected permissive cell;

(ii) culturing said transfected permissive cell for an appropriate period of time and under suitable conditions to allow the production of said viral particle;

(iii) recovering said viral particle from the cell culture; and (iv) optionally, purifying said recovered viral particle.

Preferably, the permissive cell is a mammalian cell, and more preferably a human cell. The adenoviral particles can be recovered from the culture supernatant but also from the cells after lysis and optionally can be further purified according to standard techniques (e.g., chromatography, ultracentrifugation, as described in WO 96/27677, WO 98/00524, WO 98/26048 and WO 00/50573). Moreover, the replication-competent adenoviral vector of the invention can be targeted to a particular host cell, as described above in connection with the method of the present invention.

The present invention also provides a viral particle comprising the replication-competent adenoviral vector of the invention. Such a viral particle can be prepared using the method disclosed in the previous paragraph.

The present invention also provides a host cell comprising the replication-competent adenoviral vector or infected by the viral particle of the invention. The term “host cell” as used herein refers to a single entity, or can be part of a larger collection of cells. Such a larger collection of cells can comprise, for instance, a cell culture (either mixed or pure), a tissue, an organ, an organ system, or an organism (e.g., a mammal, or the like) as described above in connection with the method of the invention. Preferably, the host cells in this context is cancerous, tumoral or hyperproliferative or prone to develop a cancer, a tumor or a hyperproliferation. It is of note that the present invention does not relate to host cells that naturally belong to the human organism and that are not isolated from the body.

The present invention also provides a composition comprising the replication-competent adenoviral vector; the viral particle or the host cell of the present invention. The composition according to the invention may be manufactured in a conventional manner for a variety of modes of administration including systemic, topical and localized administration (e.g., topical, aerosol, instillation, oral administration). For systemic administration, injection is preferred, e.g., subcutaneous, intradermal, intramuscular, intravenous, intraperitoneal, intrathecal, intracardiac (such as transendocardial and pericardial), intratumoral, intravaginal, intrapulmonary, intranasal, intratracheal, intravascular, intraarterial, intracoronary or intracerebroventricular injection. Intramuscular, intratumoral and intravenous injections constitute the preferred modes of administration. The administration may take place in a single dose or a dose repeated one or several times after a certain time interval. The appropriate administration route and dosage may vary in accordance with various parameters, as for example, the condition or disease to be treated, the stage to which it has progressed, the need for prevention or therapy and the therapeutic transgene to be transferred. As an indication, a composition may be formulated in the form of doses of between 10⁴ and 10¹⁴ iu (infectious units), advantageously between 10⁵ and 10¹³ iu and preferably between 10⁶ and 10¹² iu. The titer may be determined by conventional techniques. The composition of the invention can be in various forms, e.g., in a solid (e.g., powder, lyophilized form), or in a liquid (e.g., aqueous) form.

Moreover, the composition of the present invention can further comprise a pharmaceutically acceptable carrier for delivering said replication-competent adenoviral vector into a human or animal body. The carrier is preferably a pharmaceutically suitable injectable carrier or diluent which is non-toxic to a human or animal organism at the dosage and concentration employed (for example, see, Remington's Pharmaceutical Sciences, 16^{th} Ed., Mack Publishing Co (1980)). It is preferably isotonic, hypotonic or weakly hypertonic and has a relatively low ionic strength, such as provided by a sucrose solution. Furthermore, it may contain any relevant solvents, aqueous or partly aqueous liquid carriers comprising sterile, pyrogen-free water, dispersion media, coatings, and equivalents, or diluents (e.g., Tris-HCl, acetate, phosphate), emulsifiers, solubilizers or adjuvants. The pH of the pharmaceutical preparation is suitably adjusted and buffered in order to be appropriate for use in humans or animals. Representative examples of carriers or diluents for an injectable composition include water, isotonic saline solutions which are preferably buffered at a physiological pH (such as phosphate buffered saline, Tris buffered saline, mannitol, dextrose, glycerol containing or not polypeptides or proteins such as human serum albumin). Illustrative examples of such diluents include a sucrose-containing buffer (e.g., 1M saccharose, 150 mM NaCl, 1 mM MgCl₂, 54 mg/l Tween 80, 10 mM Tris pH 8.5) and a mannitol-containing buffer (e.g., 10 mg/ml mannitol, 1 mg/ml HSA, 20 mM Tris pH 7.2 and 150 mM NaCl).

In addition, the composition according to the present invention may include one or more stabilizing substance(s), such as lipids (e.g., cationic lipids, liposomes, lipids as described in WO 98/44143; Felgner et al., Proc. West. Pharmacol. Soc. 32 (1987), 115-121; Hodgson and Solaiman, Nature Biotechnology 14 (1996), 339-342; Remy et al., Bioconjugate Chemistry 5 (1994), 647-654), nuclease inhibitors, hydrogel, hyaluronidase (WO 98/53853), collagenase, polymers, chelating agents (EP 890 362), in order to prevent its degradation within the animal/human body and/or improve delivery into the host cell. Such substances may be used alone or in combination (e.g., cationic and neutral lipids). It may also comprise substances susceptible to facilitate gene transfer for special applications, such as a gel complex of polylysine and lactose facilitating the delivery by the intraarterial route (Midoux et al., Nucleic Acid Res. 21 (1993), 871-878) or poloxamer 407 (Pastore, Circulation 90 (1994), 1-517).

The composition of the present invention is particularly intended for the preventive or curative treatment of a cancer. The term “cancer” encompasses any cancerous conditions including diffuse or localized tumors, metastasis, cancerous polyps and preneoplastic lesions (e.g., dysplasies) as well as diseases which result from unwanted cell proliferation. In particular, the term “cancer” refers to cancers of breast, cervix (in particular, those induced by a papilloma virus), prostate, lung, bladder, liver, colorectal, pancreas, stomach, esophagus, larynx, central nervous system, blood (lymphomas, leukemia, etc.) and to melanomas and mastocytoma.

The present invention also provides a method of treating a patient suffering from a cancer or a hyperproliferative cell disorder, which comprises administering to said patient a therapeutically effective amount of the replication-competent adenoviral vector or the viral particle or the host cell of the invention. A “therapeutically effective amount” is a dose sufficient to the alleviation of one or more symptoms normally associated with the disease or condition desired to be treated. When prophylactic use is concerned, this term means a dose sufficient to prevent or delay the establishment of a disease or condition.

The method of treatment of the present invention can be used for preventive purposes and for therapeutic applications relative to the diseases or conditions listed above. The present method is particularly useful to prevent the establishment of tumors or to reverse existing tumors of any type, using an approach according to that described herein. It is to be understood that the present method can be carried out by any of a variety of approaches. Advantageously, the replication-competent adenoviral vector or the composition of the invention can be administered directly in vivo by any conventional and physiologically acceptable administration route, for example by intravenous injection, into an accessible tumor, into the lungs by means of an aerosol or instillation, into the vascular system using an appropriate catheter, etc. The ex vivo approach may also be adopted which consists in removing cells from a patient (bone marrow cells, peripheral blood lymphocytes, myoblasts and the like), introducing into the cells the replication-competent adenoviral vector of the invention in accordance with the techniques of the art and re-administering the vector-bearing cells to the patient.

According to a preferred embodiment, when the method of the invention uses a replication-competent adenoviral vector expressing a suicide gene, it can be advantageous to additionally administer a pharmaceutically acceptable quantity of a prodrug which is specific for the expressed suicide gene product. The two administrations can be made simultaneously or consecutively, but preferably the prodrug is administered after the adenovirus particle of the invention. By way of illustration, it is possible to use a dose of prodrug from 50 to 500 mg/kg/day, a dose of 200 mg/kg/day being preferred. The prodrug is administered in accordance with the standard practice. The oral route is preferred. It is possible to administer a single dose of prodrug or doses which are repeated for a time sufficiently long to enable the toxic metabolic to be produced within the host organism or the host cell. As mentioned above, the prodrug ganciclovir or acyclovir can be used in combination with the TK HSV-1 gene product and 5-FC in combination with the use of replication-competent adenoviral vectors expressing the UPRTase and/or the CDase activity as encoded by the FCY1, FURl and/or FCU1 gene.

Prevention or treatment of a disease or a condition can be carried out using the present method alone or, it desired, in conjunction with other presently available methods (e.g., radiation, chemotherapy, surgery or immunosuppressive treatment).

The present invention also provides the use of the replication-competent adenoviral vector or the viral particle or the host cell of the invention, for the preparation of a medicament for the treatment or prevention of a cancer or a hyperproliferative cell disorder by gene therapy. Within the scope of the present invention, “gene therapy” has to be understood as a method for introducing a therapeutic gene into a cell. Thus, it also includes immunotherapy that preferably relates to the introduction of a potentially antigenic epitope into a cell in order to induce an immune response which can be cellular or humoral or both.

The present invention also provides a method of enhancing the apoptotic status in a host cell, which comprises introducing in said host cell at least the replication-competent adenoviral vector or the viral particle or the host cell of the invention. In a preferred embodiment, the method is carried out in vitro. The enhancement of apoptosis can be evaluated by comparing the apoptotic status of the host cell, tissue or organism in the presence of the replication-competent adenoviral vector of the invention compared to a conventional replication-competent adenoviral vector retaining functional pIX and/or E4orf3 genes. As a result, the host cell, tissue or organism containing the replication-competent adenoviral vector of the invention is more prone to apoptosis (cell death) or is recovering less rapidly or less efficiently than a host cell, tissue or organism containing a conventional replication-competent adenoviral vector. Such an improvement of apoptosis can be determined for example by evaluating the cell death, the concentration of one or several markers that are produced in the course of apoptosis by FACS analysis (enhancement of apoptosis-associated markers by a factor of at least 2 to 10) and/or morphological analysis (e.g., enhancement of condensation of chromatin at the nuclear periphery, DNA cleavage, disassembly of nuclear scaffold proteins, formation of apoptotic bodies and/or nuclear fragmentation).

The present invention also provides the use of the replication-competent adenoviral vector or the viral particle or the host cell of the invention, for the preparation of a medicament for enhancing apoptosis (i.e., the apoptosis status) in a host cell.

The invention has been described in an illustrative manner, and it is to be understood that the terminology which has been used is intended to be in the nature of words of description rather than of limitation. Obviously, many modifications and variations of the present invention are possible in the light of the above teachings. It is therefore to be understood that within the scope of the appended claims, the invention may be practiced in a different way from what is specifically described herein.

All of the above cited disclosures of patents, publications and database entries are specifically incorporated herein by reference in their entirety to the same extent as if each such individual patent, publication or entry were specifically and individually indicated to be incorporated by reference.

### LEGENDS OF FIGURES

FIG. 1: is a schematic representation of the replication-defective adenoviral vector Ad(CMVIX). This vector retains the native pIX transcription unit at the 3′ border of the E1 deletion and further comprises the pIX coding sequence placed under the control of the early CMV promoter (hCMVp), a chimeric intron (splice) and rabbit beta globin polyadenylation sequence (poly A), and is inserted in replacement of the deleted E4 region (deletion of nt 32994 to 34998).

FIG. 2: illustrates the in vitro evaluation of the Ad5 plX-expressing replication-defective adenoviral vector Ad (CMVIX) in connection with inhibition of interferon gamma (IFNg)-induced apoptosis. A549 cells were infected with either Ad (CMVIX) or negative controls (empty E1, E3 and E4-deleted adenoviral vector or replication-defective adenoviral vector expressing plX mutant (Ad (CMVIXV117D)), 24 hours prior or concomitantly to being exposed to IFNg during 36 hours. FIG. 2A represents non-infected cells, FIG. 2B represents A549 cells infected with Ad (CMVIX) and FIG. 2C represents A549 cells infected with Ad (CMVIXV117D). Morphological criteria of apoptotic cell death were evaluated in Epon sections. Arrows point to pIX-induced clear amorphous inclusions. Bar 1 μm

The following examples serve to illustrate the present invention.

### EXAMPLES

The constructions described below are carried out according to the standard techniques of genetic engineering and molecular cloning detailed in Sambrook et al. (Molecular Cloning, A Laboratory Manual, Cold Spring Harbor Laboratory Press, Cold Spring Harbor N.Y. (2001)). The cloning steps employing bacterial plasmids are performed in Escherichia coli (E. coil) strain 5K or BJ, whereas those employing M13-based vectors are carried out in E. coil NM522. PCR amplification is performed according to standard procedures, as described in PCR-Protocols. A guide to methods and applications (edited by Innis, Gelfand, Sninsky and White, Academic Press Inc (1990)). The adenoviral fragments used in the constructions described hereinafter are indicated according to their position in the Ad5 genome as disclosed in Chroboczek et al., (Virology 186 (1992), 280-285) or in the GeneBank data bank under the reference M73260. All viral genomes were constructed as infectious plasmids by homologous recombination in Escherichia coil between a transfer plasmid and a Pacl-linearized plasmid containing the viral backbone as described in Chartier et al., (J. Virol. 70 (1996), 4805-4810). Cells are cultured according to standard procedures or to the manufacturer's recommendations.

### Materials and Methods

Cells and Viruses:

Monolayer human lung carcinoma A549 cells (Smith, American Review of Respiratory Disease 115 (1977), 285-293; ATCC CCL-185), 293 cells (Graham et al., J. Gen. Virol. 36 (1977), 59-72; ATCC CRL-1573) and 293-E40RF6/7 cells (Lusky et al., J. Virol. 72 (1998), 2022-2032) were grown in Dulbecco's medium supplemented with 10% fetal calf serum (FCS). Cells were infected at 80% confluency with the different adenoviruses (wild type (wt) AdS, pIX VI 17D mutated AdS or Ad vectors) at a multiplicity of infection (MOI) of 20 PFU per cell in 2% serum. A549 cells were transfected by calcium phosphate co-precipitation as previously described (Chen, Mol. Cell Biol. 7 (1987), 2745-2752).

Ad Vectors:

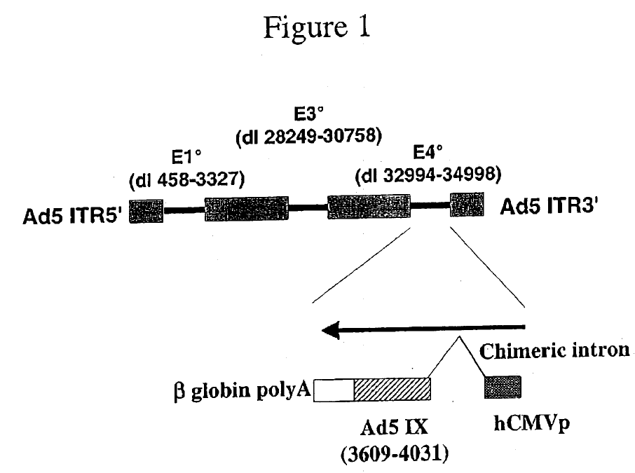

Ad (CMVIX) is illustrated in FIG. 1. It was obtained from the E1, E3 and E4-deleted AdTG9546 vector (Lusky et al., J. Virol. 72 (1998), 2022-2032; E1 deletion from nt 459 to nt 3327, E3 deletion from nt 28249 to nt 30758 and E4 deletion from nt 32994 to nt 34998), and contains in replacement of the E4 deleted region the Ad5 pIX encoding sequence (nt 3609 to 4031) under the transcriptional control of the human CMV (hCMV) promoter, a chimenc intron (found in the pCI vector available from Promega comprising the human beta globin donor splice site fused to the immunoglobulin gene acceptor splice site) and the polyadenylation signal from the rabbit beta-globin gene (nt 1542 to 2064 of the sequence disclosed in the GeneBank data bank under the reference K03256). The Ad5 pIX coding sequence was amplified by PCR using oligonucleotides that contain Sall and EcoRV sites at their 5′ and 3′ extremities, respectively, which allowed the directed cloning into the polylinker of the expression cassette (5′ oligonucleotide: 5′-GAATTCGTCGACCCATGAGCACCAACTCG-3′ (SEQ ID NO: 1) and 3′ oligonucleotide: 5′-GAATTCGATATCTTAAACCGCATTGGGAGGGGAGG-3′ (SEQ ID NO: 2)). After sequencing, the product of amplification was subcloned into the transfer plasmids. The Ad5 pIX expression cassette was flanked by adenovirus sequences required for homologous recombination in the E4 region.

Ad (CMVIXV117D) is similar to Ad (CMVIX) with the exception that it expresses the plX mutant VI 17D instead of wild-type pIX, under the control of the CMV promoter. VI 17D is a pIX mutant in which the valine residue (V) in position 117 was substituted by an aspartic acid (D) (“QuickChange site-directed mutagenesis” system: Stratagene), resulting in the disruption of the C-terminal coiled-coil domain of wt pIX (Rosa-Calatrava et al., J. Virol. 75 (2001), 7131-7141).

Ad (CMVIXV117D) and Ad (CMVIX) were grown on 293-E40RF6/7 cells. Virus propagation, purification and titration of infectious unit (IU/ml) by indirect immunofluorescence of the DNA binding protein (DBP) were as described in Lusky et al., (J. Virol. 72 (1998), 2022-2032).

Recombinant Eukaryotic Expression Vectors:

The pIX coding sequence was mutated by introducing either short deletions (del 13-15, del 22-23, del 26-28, del 63-70) or point mutations (Q106K, E113K, L114P, V117D and L-V), as previously described (Rosa-Calatrava et al., J. Virol. 75 (2001), 7131-7141). For example, the various mutated pIX sequences were introduced into three types of expression vectors, pXJ41 plasmid (Rosa-Calatrava et al, J. Virol. 75 (2001), 7131-7141), the pM plasmid and the VPi6 plasmid (CLONTECH, Palo Alto, Calif.) for expression as fusion proteins in the N-terminal region with the GAL4 DNA binding domain and the VP16 transactivation domain, respectively.

The plasmid pSG5 expressing the wild-type 69 kDa isoform PML (PML3B, accession number M80185) and corresponding mutants in the RING finger (Q59C60-E59L60) or in the coiled-coil domain (P1:del 216-33) were previously described (De The et al., Cell 66(1991), 675-684). The sequence encoding the wt or the mutated 69 kDa isoform PML protein was also introduced in frame into pM and pVP16 plasmids for expression in fusion with the GAL4 DNA binding domain and the VP16 transactivation domain, respectively (Sternsdorf et al., J. Cell Biol. 139 (1997), 1621-1634).

The G0-TK-CAT reporter (Webster et al., Cell 52 (1988), 169-178) contains the CAT gene driven by the HSV-1 thymidine kinase (TK) promoter (−105/+51) and bears a single GAL4 binding site inserted 5′ to the TK promoter. The TATA box (TATTAAG) was mutated to a TGTA box (TGTAAAG) using the “Quick Change site-directed mutagenesis” system (Stratagene). All the constructions were verified by DNA sequencing.

Antibodies:

Rabbit polyclonal anti-pIX and anti-Ad5 penton-base antibodies were previously described (Rosa-Calatrava et al., J. Virol. 75 (2001), 7131-7141). Chicken anti-PML, rabbit anti-PML (De The et al., Cell 66 (1991), 675-684), anti-SP100 (De The et al., Cell 66 (1991), 675-684); and anti-hexon (Valbiotech, Paris) antibodies have been previously described (Puvion-Dutilleul et al., Experimental Cell Research 218 (1995), 9-16 and Puvion-Dutilleul et al., Biology of the Cell 91(1999), 617-628). Monoclonal anti-fiber (Legrand et al., J. Virol, 73 (1999), 907-919) were previously described. Monoclonal anti-PML (PMG3) and anti-SUMO (anti-GMPl) antibodies were purchased from Stratagene and Zymed, respectively.

Electron Microscopy:

Fixation and Embedding:

Monolayers of A549 cells were infected with Ad5 wt or mutated AdIX/V117D (see above). After 30 mm virus adsorption, the cells were rinsed with PBS, fresh medium was added and the incubation was prolonged for 18 or 28 h post-infection (pi), before fixation.

For conventional studies, cells were fixed with 1.6% glutaraldehyde (Taab Lab. Equip. Ltd, Reading, UK) in 0.1 M PBS for 1 h at 4° C. During the fixation step, cells were scraped from their plastic substratum and centrifuged. The resulting pellets were rinsed in the above-mentioned buffer, dehydrated in increasing concentrations of ethanol and embedded in Epon. Ultrathin sections were collected on Formvar-carbon-coated gold grids (mesh 200) and stained with uranyl acetate and lead citrate prior to observation with a Philips 400 transmission electron microscope, at 80 kV, at 13 000 magnification.

For immunogold detection of antigens, cell cultures were fixed with 4% formaldehyde (Merck, Darmstadt, Germany) instead of glutaraldehyde, dehydrated in methanol and embedded in Lowicryl K4M (Polysciences Europe Gmbh, Eppelheim, Germany) instead of ethanol and Epon, respectively. Polymerisation of Lowicryl-embedded samples was carried out under long wavelength UV light (Philips TL 6W fluorescent tubes) at −30° C. for 5 days and subsequently at room temperature for 1 day. Ultrathin sections were collected on Formvar-carbon-coated gold grids (mesh 200) and processed for immunocytology prior to uranyl acetate staining.

Immunocytology:

Grids bearing Lowicryl sections were floated for 2 min over drops of Aurion BSA-C (purchased from Biovalley, France) (0.01% in PBS) in order to prevent background, prior to be incubated for 30 mm on 5 μl drops of primary antibody diluted in PBS as follows: rabbit anti-pIX (1/50), anti-fiber (1/50) or anti-penton-base (1/50) antibodies for 30 mm, rabbit anti-PML (ZINA) (1/10) or anti-SP100 (1/20) antibodies for 1 h, goat anti-hexon (1/200) antibodies for 30 mm. After washing over PBS drops, the grids were incubated for 30 mm over 511 drops of secondary antibody diluted 1/25 in PBS: either goat anti-rabbit IgG and/or IgM or goat anti-mouse IgG (British Biocell International LTD, Cardiff, UK) or monkey anti-goat IgG (Valbiotech, Paris, France), conjugated to gold particles, 10 nm in diameter. After rapid passages over PBS drops, the grids were washed in a stream of distilled water, air-dried, and finally, routinely stained with uranyl acetate prior to observation. For controls, it was verified that the primary antibodies raised against viral proteins did not react with cellular material (from non-infected cells) and that the secondary antibodies did not bind non-specifically to viral material.

In Situ Hybridization:

In order to localise viral RNA, in situ hybridisation was performed on Lowicryl sections using a commercial biotinylated genomic probe (Enzo Biochemicals Inc., New York, USA), as previously described (Puvion-Dutilleul, et al., Biology of the Cell 91 (1999), 617-628). Briefly, sections were digested with DNase 1 (1 mg/ml, 1 h, Worthington Biochemical Corp. Freehold, USA) prior to the hybridisation step in order to eliminate the viral single-stranded DNA. To tentatively unmask the viral RNA of the sections which are hidden by proteins, some sections were incubated in the presence of a protease solution prior to DNase digestion. Hybridization was performed for 90 mm at 37° C. in a moist chamber. Hybrids were subsequently detected using anti-biotin antibody conjugated to gold particles, 10 nm in diameter (British Biocell International, Cardiff, UK). Finally, the grids were stained with uranyl acetate.

Immunofluorescence:

Immunofluorescence staining experiments were carried out as previously described (Rosa-Calatrava et al., J. Virol. 75 (2001), 7131-7141).

Primary antibodies were diluted in PBS containing 0.1% Triton X-100. The anti-pIX rabbit polyclonal antibody was used as previously described (Rosa-Calatrava et al., J. Virol. 75 (2001), 7131-7141). Rabbit polyclonal anti-SP10O, chicken anti-PML, monoclonal mouse anti-PML (PMG3) and anti-SUMC (anti-GMP1) were diluted respectively at 1/5000, 1/250, 1/100 and 1/100 in PBS containing 0.1% Triton X-100. After incubation for 1 hour, the coverslips were washed several times in PBS-0.1% Triton X-100 and then incubated with goat Cy3 or Cy5-conjugated anti-mouse IgG and/or donkey Cy3 or FITC-labeled anti-rabbit IgG and/or donkey Cy3 antichicken(Sigma), at concentrations recommended by the suppliers.

Nuclei were then counter-stained with Hoechst 33258. After staining, the coverslips were mounted and cells were analyzed with a confocal laser scanning microscope (Leica). Image enhancement software was used to balance signal strength and 8-fold scanning was used to separate signal from noise.

### Example 1

Distribution and Evolution of pIX-Induced c.a. Inclusions in Ad5-Infected Cells:

It was previously shown that, independently of the other viral proteins, pIX induces the formation of characteristic nuclear structures, designated as clear amorphous (c.a.) inclusions (Rosa-Calatrava et al., J, Virot. 75 (2001), 7131-7141). In order to (i) more precisely examine the intranuclear distribution of pIX, and (ii) further underline the putative function of associated inclusions in the overall context of infection and to characterize Ad-induced alterations of the host nuclear ultrastructure, Ad5-infected A549 cells were analyzed by immuno-electron-microscopy (immuno-EM) and immuno-fluorescence (IF) with anti-pIX polyclonal antibodies. Alteration of the nuclear morphology occurs in three major steps following Ad infection: an early step concomitant with viral DNA replication, an intermediate step taking place at about 18 h pi and a late step at about 24-28 h pi.

Low amounts of pIX is detected in the cytoplasm (45 mm pi) and nuclei (up to 4 h pi) of early infected cells, corresponding to polypeptides released from the capsid of the infecting viruses. After this initial period, no significant pIX labeling could then be observed until 12-14 h pi, corresponding to the onset of viral DNA replication and consistent with the low level of pIX transcription at this stage.

In the intermediate phase of infection, a slight labeling of the fibrillo-granular network by anti-pIX staining is at first observed, probably corresponding to pIX molecules engaged in viral gene transactivation (around 16 h pi). Such a localization still remains persistent during the complete late phase of infection. Once neosynthesized, pIX progressively accumulates in the host nucleus and induces the formation of specific structures (c.a. inclusions) which become visible as irregularly shaped patches, dispatched (over-spreaded) within the overall fibrillo-granular network (see also Rosa-Calatrava et al., J. Virol. 75 (2001), 7131-7141). They are easily identifiable by their sole morphology in EM analysis; up to 1 μm in diameter, they look like some roundish and homogeneous inclusions with apparent weak density to electron transmission. In addition to the c.a. inclusions, Ad infection induces other types of structure negative for pIX staining, of yet unknown function: (i) amorphous electron opaque inclusions (o.i.) which are strongly labelled with antibodies against pIVa2, the product of the intermediate gene Iva2 (Lutz and Kedinger, J. Virol. 70 (1996), 1396-1405), compact rings which contain non-polyadenylated viral RNA (Puvion-Dutilleul et al., J. of Cell Science 107 (1994), 1457-1468) and replication foci (Puvion-Dutilleul and Puvion, Biology of the Cell 71 (1991), 135-147). Each of the pIX-containing c.a. inclusions is intensively and homogeneously labeled with the anti-pIX antibodies, while all of the other virus-induced or host cellular structures are negative for pIX staining, except, as known, crystals of capsidic proteins and virions.

pIX-containing c.a. inclusions show a precise dynamic evolution and continuously grow in size during the late phase of infection. The accumulation of inactive viral genomes and crystalline arrays of virus particles (Puvion-Dutilleul and Pichard, Biol. Cell 76 (1992), 139-150 arid Puvion-Dutilleul et al., Journal of Structural Biology 108 (1992), 209-220) progressively induces their exclusion from the central fibrillo-granular viral region, and their redistribution within the perinuclear translucent area of the nucleus. In good agreement with immuno-EM observations, IF-staining experiments show an evolution from a “micro-speckled” pattern of pIX distribution to a “macro-speckled” aspect, as the infection progresses into the late phase. At the later stage of infection (beyond 28 h pi), c.a. inclusions seem to coalesce and form bright structures of accumulation. Sometimes two or three of these inclusions can be observed self-juxtaposed within the perinuclear transluscent area (see below). At 36 h pi, many c.a. inclusions are observed in the cytoplasm, superimposed on a diffuse cytoplasmic pIX staining.

EM and IF immunostaining were also performed with cells infected with pIX-V117D Ad5 expressing the V117D variant of pIX. Whereas the mutated pIX is still incorporated into virions, our observations reveal the absence of c.a. inclusions and a subsequent diffused localization of pIX V117D within the cytoplasm, the nuclear fibrillo-granular network and the perinuclear transluscent area. This supports that the integrity of the coiled-coil domain of pIX is required for the formation of c.a. inclusions, likely mediated through self-multimerisation.

### Example 2

The pIX-Induced c.a Inclusions Exhibit No Transcriptional, Splicing or Viral Encapsidation activities:

The above-described experiments support (i) c.a. inclusion formation via an active process of pIX self-assembly, (ii) their specific nuclear retention, (iii) their determined temporal appearance and dynamic, (iv) the importance for their size and number during the late phase of infection. On this basis, it was important to identify whether viral functions are also linked to c.a. inclusions.

Previous studies have revealed that pIX is a transcriptional activator (Lutz et al., J. Virol. 71(1997), 5102-5109), probably interacting through its coiled-coil domain with components of the transcriptional cellular machinery (Rosa-Calatrava, I Virol. 75 (2001), 7131-7141) and likely contributing to the program of Ad gene expression. The coiled-coil domain of the pIX protein also plays a central role in the formation of c.a. inclusions. As discussed above, (I) in c.a. inclusions, no viral RNA was detected by in situ hybridization experiments, whereas, as expected, the fibrillo-granular network, that is active in viral transcription, as well as the clusters of interchromatin granules and the cytoplasm were labeled; (ii) pIX mutants exclusively retaining the transactivation function or the capacity to form c.a. inclusions were isolated; (iii) during the late phase of infection, c.a. inclusions were progressively excluded from the transcriptionally active granulo-fibrillar network and were relegated to the nuclear periphery, into the electron-translucent area, over to the cytoplasm. Moreover, RNA polymerase II was also undetectable in the c.a. inclusions, although it was found associated to the fibrillo-granular network and the cluster of interchromatin granules (data not shown).

All together, these observations rule out any linkage of pIX transcriptional activity with the c.a. inclusions. On this basis, a temporal dissociation of the transcriptional and the c.a. inclusions properties of pIX is expected during Ad infection.

Late in infection, viral RNA processing monopolizes the host cell splicing machinery, a process which morphologically results in the disappearance of two cellular structures, the coiled bodies (Rebelo et al., Molecular Biology of the Cell 7 (1996), 1137-1151) and the interchromatin granule-associated zone (Besse et al., Gene Expression 5 (1995), 79-92). Splicing events remain associated with the viral-induced fibrillo-granular network and with clusters of interchromatin granules (Puvion-Dutilleul et al., Journal of Cell Science 107 (1994), 1457-1468). Looking for splicing-related events within pIX-induced c.a. inclusions, the cellular distribution of spliceosome components was reexamined: U1 and U2 snRNAs, SnRNPs, viral transcripts (as mentioned above) or poly(A)+RNA: they were all located in clusters of interchromatin granules of late infected nuclei, but none of them was detected within c.a. inclusions. Together with the fact that no pIX-specific labeling could be found in the interchromatin granules, these results clearly indicate that pIX and c.a. inclusions are not involved in post-transcriptional processes during infection.

As pIX is a structural protein which stabilizes the interactions within the Ad capsid (Colby and Shenk, J. Virol. 39 (1981), 977-980; Furcinitti et al., EMBO J. 8 (1989), 3563-3570; Ghosh-Choudhury et al., EMBO J. 6 (1987), 1733-1739), it was also examined whether major capsid proteins are co-localized within the c.a. inclusions. Immunostaining shows that c.a. inclusions are weakly labeled with anti-hexon antibodies, and entirely devoid of penton base and fiber proteins, as revealed by the absence of labeling with corresponding antibodies. By contrast and as expected, an intense labeling was generated with anti-hexon, anti-penton base and anti-fiber antibodies over the viruses and protein crystals. Consistent with the absence of viral DNA (determined by in situ hybridization) and virions in c.a. inclusions, these results clearly indicate that pIX-induced c.a. inclusions are not involved in the process of virion encapsidation.

It appears therefore that pIX-induced c.a. inclusions are completely unrelated to the essential viral processes represented by DNA transcription, RNA splicing and virion assembly. Consistent with these results, none of the viral structures supporting these activities seems to be modified or altered in the context of infection by Ad5 IX/V117D. One may presume that c.a. inclusions might be implicated in the alteration of the host cellular metabolism resulting from viral infection.

### Example 3

Host Cellular PML and SP100 Proteins are Detected Within the c.a. Inclusions During the Late Phase of Infection:

Immuno-EM using either monoclonal or polyclonal anti-PML antibodies, stained by immunogold anti-pIX staining, indicate that c.a. inclusions clearly contain both PML and SP100 proteins from their very initial stage of formation (at 16-17 h pi), until they finally constitute large perinuclear inclusions, late in infection (28 or 36 h pi). Interestingly, while the c.a. inclusions were always intensively and homogeneously stained with anti-PML and anti-SP100 antibodies during infection, immuno-EM revealed that all the other late nuclear viral compartments were only poorly or not labeled (e.g., the fibrillo-granular and inter-chromatin granular zones). These data support specific association of PML and SP100 cellular proteins with the pIX-induced c.a. inclusions.

The presence of these two constitutive components of the PML nuclear domains (also referred to as FML oncogenic domains, PODs), within c.a. inclusions cannot be just fortuitous. Therefore, it was then explorated whether pIX was directly implicated in the process of alteration of these host nuclear domains promoted by adenovirus infection.

### Example 4

Ad Infection Induces Late Confinement of Endogenous PML Protein Within the pIX-Induced c.a. Inclusions:

It was previously shown that, during the early phase of adenovirus (Ad) infection, PODs are disrupted by the AdE4orf3 gene product which redistributes PML protein into a meshwork of viral fibrous-tracks structures (Carvalho et al., J. Cell Biol. 131 (1995), 45-56; Doucas et al., Genes Dev. 10 (1996), 196-207; Puvion-Dutilleul et al., Exp. Cell Res. 218 (1995), 9-16). However, the fate of PML localization during the late phase of infection has yet been unexplored. For this purpose, IF-staining of Ad5 wt-infected cells was performed at various times post infection (pi) in order to visualize the entire dynamics of PML nuclear distribution during the course of infection.

It was observed that pIX-induced c.a. inclusions that are formed in the host nucleus most often appear co-localized with the (E4orf3-induced) PML-containing fibrous tracks or were found within their immediate vicinity. While pIX accumulates in the infected cells and the c.a. inclusions grow in size, the PML-containing fibrous tracks progressively vanish to finally become undetectable in the late stage of adenoviral infection.

In order to test whether the progressive loss of PML immunoreactivity is caused by the degradation of the protein in the c.a. inclusions or by nuclear redistribution, the presence of PML protein in extracts of infected cells was analyzed by Western-blotting. For this purpose, after pretreatment with IFNg during 24 h (to increase endogenous expression of PML as reported by Stadler et al., Leukemia 9 (1995), 2027-2033), A549 cells were infected with wt Ad5 at relative high MOI (50 pfu) for several times until 72 h pi.

In non-infected cells, IFNg treatment induces the synthesis of different modified forms and high-molecular-weight isoforms of PML, having molecular weights ranging from 80 to 130 kDa. Cells infected with wt AdS apparently exhibit the same pattern of PML protein as non-infected cells, even at 72 h pi, although a decrease of the total PML signal as well as a decrease of pIX and cellular actin is observed after 60 h pi, said decreases correlating with a loss of cell material due to cell lysis at the late stage of adenoviral infection. These results indicate that PML proteins are not degraded during adenoviral infection and that the adenovirus-induced disruption of PODs is not associated with a degradation of their organizer protein, PML. These results are corroborated by the above-described EM observations which reveal a persistent detection of a PML signal within the c.a. inclusions, even after 48 h pi. The paradoxical results obtained by the IF and the EM experiments can likely be explained by the fact that the PML protein might be inaccessible to antibodies inside the inclusions (thus non detectable by IF-immunostaining), unless exposed at the surface of the nuclear slice (thus detectable by immuno-EM analysis). This hypothesis raises the possibility of a confinement of PML protein within the pIX-induced c.a. inclusions, and is supported by IF- and EM analyses of cells infected with pIX-mutated Ad5 (Ad IXV117D). This mutant of an Ad5 vector is deficient of inducing the formation of c.a. inclusions and does not show a co-localization of c.a. inclusions with E4orf3-induced fibrous tracks.

These results support that, concomitantly with pIX accumulation, PML is progressively deviated from its primary E4orf3-induced location and sequestered inside the c.a. inclusions. Interestingly, SP100, another POD-related protein, is also recruited to the c.a. inclusions, with a time course similar to that of PML, as revealed by immuno-EM. These observations strongly suggest that the presence of POD components within the pIX-induced c.a. inclusions may reflect a specific adenoviral strategy designed to interfere with POD-related cellular functions during the infectious cycle.

### Example 5

Recombinant pIX Protein Induces the Formation of c.a. Inclusions Specifically Over Endogenous PODs, but Without Disrupting Them:

In order to validate the above hypothesis, the intrinsic pIX properties were examined with respect to the integrity of cellular PML protein and associated PODs. For this purpose, the recombinant wt pIX protein was overexpressed in transfected cells in a non-viral context, i.e., from a plasmid vector. Immunofluorescence staining shows that pIX accumulates and induces the formation of c.a. inclusions over (i.e., on or in close proximity to or in the area of) the endogenous PML nuclear domains up to completely swallowing them. A persistent, “dots-like” co-localization of PML and other POD constitutive components, like SP100- and SUMO-1 proteins, with the c.a. inclusions shows that the POD components are not subject to any nuclear redistribution. Moreover, the stable detection of the POD components as soon as 48 h post-transfection suggests that the POD components are not degraded in the c.a. inclusions.

pIX mutants (Rosa-Calatrava et al., J. Virol. 75 (2001), 7131-7141) were evaluated for their ability to induce the formation of c.a. inclusions that swallow POD nuclear structures. A549 cells were transfected each by one of a series of pIX mutant-encoding plasmids and the resulting cells were tested by immunofluorescence staining using polyclonal anti-pIX and monoclonal anti-PML antibodies. An accumulation over POD could not be detected in cells producing pIX mutants being altered in the coiled-coil domain (these mutations also abolish the formation of c.a. inclusions and result in a diffuse cytoplasmic and nucleoplasmic distribution as shown above). Similarly, modifications of the net charge of the coiled-coil domain completely or partially abolish pIX-accumulation on or in the area of PODs. In marked contrast, mutations affecting either the N-terminal or central domains of the protein do not alter this process. These results clearly establish that the integrity of the coiled-coil domain of pIX is essential for the co-localization of pIX with PODs and for embedding them in pIX.

These observations demonstrate that pIX is unable by itself to disrupt endogenous PML nuclear domains, but specifically accumulates together with them. Moreover, there is a good correlation with the above data concerning the association of c.a. inclusions with the host nuclear matrix, suggesting a strong link between the formation of c.a. inclusions in the nucleus and their specific accumulation on or in close proximity to POD, which are nuclear matrix-linked bodies: both processes are dependent on the integrity of the coiled-coil domain of pIX.

### Example 6

PML and pIX Proteins Interact Via their Coiled-Coil Domains:

The PML protein is the structural organizer of PODs by constituting a concentric multilayered meshwork at the periphery of them. In this context and consistent with the above results, PML could be a preferred target for the pIX protein to drive the formation of c.a. inclusions, suggesting a putative affinity between both proteins. In order to verify this hypothesis, the distribution of recombinant PML with reference to pIX protein was investigated using transiently co-transfected cells. Immunostaining shows that PML forms a nuclear pattern of large dots, corresponding to enlarged PODs, which are partially co-localized or juxtaposed with pIX-induced c.a. inclusions. EM analysis reveals that corresponding structures share common domains. In this context, pIX is detected within the concentric multilayered meshwork of PML, as well as PODs and c.a. inclusions. In contrast, a deletion of the predictive coiled-coil domain of PML, which was previously shown to abolish homo-oligomerization of the protein and to induce a diffused nuclear pattern of the variant, abolishes co-localization with pIX-induced inclusions. On the other hand, point mutations in zinc-binding domains of the PML protein, including RING finger and B boxes (De The, Cell 66 (1991), 675-684; Borden et al., EMBO J, 14 (1995), 1532-1541; Borden et al., Proc. Natl Acad. Sci. USA 93 (1996), 1601-1606), which were previously Shown to prevent the formation of mature PODs, but fairly induce aggregates of PML, do not alter the co-localization with pIX-induced c.a. inclusions. Similar results were obtained with different PML isoforms provided that in these isoforms the putative coil-coiled domain was held upright.

These results clearly suggest that there is a specific affinity between pIX and PML, which seems to depend on the integrity of their respective coiled-coil domains. It should be noted that both domains are rich in hydrophobic residues and are known to drive heteromeric interactions between proteins.

In order to determine whether pIX directly interacts with the PML protein, a two-hybrid assay system was carried out in human A549 cells. For this purpose the cells were co-transfected with plasmids encoding pIX and PML fused with the GAL4 DNA binding domain and the VP16-transactivating domain, respectively. The fusion of the Gal4 or the VP16 domain was made with the N-terminus of pIX in order to keep the C-terminal coiled-coil domain of pIX freely accessible.

The cells were then transfected with a mutated 64-TK-CAT reporter plasmid, which contains the CAT gene driven by the HSV-1 thymidine kinase (TK) promoter and which bears a single GAL4 binding site inserted 5′ to the TK promoter (Webster et al., Cell 52 (1988), 169-178). The TATA box (TATTAAG) of the TK promoter was mutated into a TGTA box (TGTAAAG) to prevent the TATA-specific transactivating activity of pIX (as described in Lutz et al., J. Virol. 71 (1997), 5102-5109). Immunoblotting assays were performed on the selected clones to verify that equal levels of the pIX and the PML fusion proteins are co-produced. CAT activities were measured in order to evaluate the capability of the pIX fusion protein to interact with the PML fusion protein. In contrast to negative controls, a significant signal is detected for cells co-expressing the fusion of the GaL4 DNA binding domain with wt pIX in combination with the VP16-PML fusion. The expression of the fusion protein combining the Gal4 DNA binding domain with a pIX mutant in which the coiled-coil domain is mutated (e.g., VI 17D and E113L) does not result in a significant CAT activity, when co-expressed with the V16-PML fusion. On the other hand, the expression of GAL4 fusion proteins with pIX mutants in which the N-terminal domain is mutated (e.g., del 22-23) leads to a similar CAT activity as the GAL4-wt pIX fusion when co-expressed with the V16-PML fusion.

In the same way, the co-expression of the Gal4-wt pIX fusion protein together with the fusion protein combining the VP16 transactivating domain with PML mutants in which the coiled-coil domain is deleted, does not result in a significant CAT activity, in comparison with positive controls.

These results strongly support that PML and pIX are capable of heteromeric interaction, which likely occurs via their respective putative hydrophobic coiled-coil domains. Interestingly, whereas like PML, SP100 protein is redistributed within pIX induced c.a. inclusions during the late phase of infection (see above), no interaction between SP100 and pIX proteins could be detected using the above-described two-hybrid assay system.

### Example 7

Arsenic Treatment of Cells Fails to Disrupt PODs When they are Confined to pIX-Induced c.a. Inclusions:

Arsenic treatment during a few hours induces the targeting of the nucleoplasmic fraction of PML to the matrix-bound PODs, but a prolonged exposure leads to its degradation and the subsequent disappearance of these nuclear domains (Zhu et al., Proc. Natl. Acad. Sci. USA 94 (1997), 3978-3983). To evaluate the effect of PODs' confinement into pIX-induced c.a. inclusions, A549 cells were transfected with plasmids encoding either wt pIX or pIX mutants and concomitantly treated with arsenic. These cells were analyzed by immunofluorescence staining assays using polyclonal anti-pIX and monoclonal anti-PML antibodies. The results show that, when pIX induces the formation of c.a. inclusions in co-localization with PODs, arsenic treatment fails to induce their complete disappearance, in contrast to the effect observed in non-transfected cells exposed to arsenic. The observed dot-like pattern of PML corresponds to remaining PODs, and similar observations occur with SF100- or SUMO-1-specific stainings. If pIX mutants that are altered either in their N-terminal or in their central region are expressed in arsenic-treated cells, a similar protection of PODs against arsenic exposure is observed. In contrast, the expression of pIX mutants which are mutated in the coiled-coil domain, abolishes the formation of nuclear c.a. inclusions and does not prevent the arsenic-induced disappearance of PODs.

These results clearly demonstrate the intrinsic property of pIX-induced c.a. inclusions to confine host PODs in a non-viral context. Such an activity seems to be permitted by the heteromeric interaction between pIX and PML. It is postulated in the context of the present invention that a similar function of c.a. inclusions, i.e., a confinement of PODs in c.a. inclusions, may also occur during Ad infection, since, as has already been shown, wt pIX mainly accumulates on or in close proximity to PML-containing fibrous tracks and accumulate and sequester PML protein into c.a. inclusions.

It is one strategy of the adenoviruses in the infection cycle to alter in a permanent manner the PML nuclear domains. This strategy appears to be different form those adopted by other DNA viruses like HSV or CMV. Instead of an early degradation of the PML protein, adenovirus seems to induce a primary de-localization of PML, initiated by the early E4orf3 product, followed by a further re-laying and sequestration supported by pIX during the late phase of infection, via a putative confinement of the PML protein within c.a. inclusions.

### Example 8

Overexpression of wt pIX Interferes with Interferon-Induced Apoptosis:

A549 cells were infected with either Ad (CMVIX) or negative controls 24 hours prior to or concomitantly with being exposed to IFNg during 36 hours. Negative controls are an empty E1, E3 and E4 deleted adenoviral vector and Ad (CMVIXV117D) expressing a pIX mutant (PIXV117D) which is unable to form c.a. inclusions on or in close proximity to host PODs. Morphological criteria of apoptotic cell death (condensation of chromatin, cleavage of DNA, disassembly of nuclear scaffold proteins, formation of apoptotic bodies and nuclear fragmentation, as described by Kerr et al., Br. J. Cancer 26 (1972), 239-257) were evaluated in Epon sections for every case.

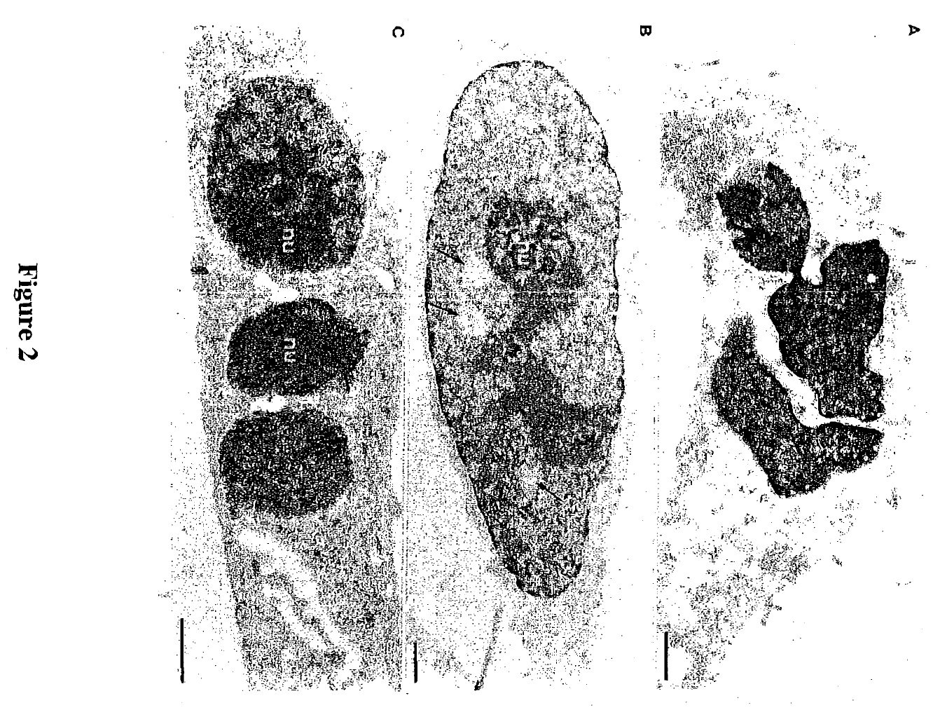

As shown in FIG. 2, non-infected cells (FIG. 2A) showed a fragmented nucleus and the condensed chromatin is pronounced. Two nuclear lobes are interconnected by a narrow strand of nucleoplasm. FIG. 28 represents A549 cells infected with Ad (CMVIX) expressing the wt Ad5 pIX protein. Oval nuclei were observed with condensed chromatin mainly restricted to a thin perinuclear layer, whereas a fine chromatin fills the nucleoplasm. The three usual components of the large nucleolus (flu): the fibrillar centers, the surrounding dense fibrillar component and the granular component, are easily recognizable. Arrows point to pIX-induced clear amorphous inclusions. FIG. 20 shows A549 cells infected with Ad (CMVIXV117D) expressing the pIX mutant V117D. The nucleus is highly lobed and, in this section, gives the appearance of being fragmented. The condensed chromatin is distributed largely within the lobes. The nucleoli (nu) are compact.

In conclusion, following IFNg treatment, uninfected cells and cells infected by E1, E3 and E4-deleted adenoviral vector or vector expressing the pIX-V117D mutant showed fragmented nuclei which, depending on the plane of the section, took the appearance of individual lobes or of lobes interconnected by a narrow strand of nucleoplasm. The condensed chromatin was widely distributed within the lobes. These cells clearly present morphological characteristics of apoptosis.

In contrast to this, cells infected with Ad (CMVIX) expressing Ad5 wt pIX exhibited oval nuclei with a condensed chromatin restricted to a thin layer at the nuclear border. The nucleoli were large and similar to those observed in untreated cell cultures, Indeed, their three compartments (fibrillar centers, the surrounding dense fibrillar component, and the granular component) were clearly visible. These cells also showed at the cut surface the presence of one or several pIX-induced clear amorphous inclusions located in the nucleoplasm. None of cells overexpressing wt Ad5 pIX present morphological characteristics of apoptosis. It appears that the absence of a fragmented nucleus and of abundant condensed chromatin is probably the result of the synthesis of Ad5 pIX in the host cell.

Each patent, patent application and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A method of modulating one or more cellular activitie(s) dependent on a POD nuclear structure in a host cell, comprising contacting a molecule of adenoviral origin with said POD nuclear structure, wherein said molecule of adenoviral origin is capable of interacting with said POD nuclear structure.

2. The method of claim 1, which comprises introducing in said host cell at least a molecule of adenoviral origin, wherein said molecule of adenoviral origin provides a reduction or an inhibition of one or more cellular activitie(s) dependent on a said POD nuclear structure.

3. The method of claim 2, wherein said host cell is infected by a virus and wherein said adenoviral molecule provides a reduction or an inhibition of the antiviral cellular activity dependent on said POD nuclear structure.

4. The method of claim 3, wherein said virus is a replication-defective adenoviral vector.

5. The method of claim 4, wherein said replication-defective adenoviral vector is deficient for E1 and E4 functions, and optionally for E3 function.

6. The method of claim 4 or 5, wherein said replication-defective adenoviral vector further comprises a transgene.

7. The method of claim 1, wherein said molecule of adenoviral origin is a polypeptide capable of providing a reduction or an inhibition of one or more cellular activities dependent on said POD nuclear structure.

8. The method of claim 1, wherein said molecule of adenoviral origin is a nucleic acid sequence encoding a polypeptide capable of providing a reduction or an inhibition of one or more cellular activities dependent on said POD nuclear structure.

9. The method of claim 7 or 8, wherein said polypeptide of adenoviral origin is selected from the group consisting of pIX and E4orf3, taken individually or in combination.

10. The method of claim 8, wherein said nucleic acid sequence encoding a polypeptide of adenoviral origin is carried by said replication-defective adenoviral vector.

11. The method of claim 10, wherein said nucleic acid sequence encoding a polypeptide of adenoviral origin is inserted in said replication-defective Ad in replacement of the deleted E4 region and wherein said transgene is inserted in replacement of the deleted E1 region.

12. The method of claim 11, wherein said nucleic acid sequence encoding a polypeptide of adenoviral origin and said transgene are transcribed in antisense orientation to each other.

13. The method of claim 8, wherein said nucleic acid sequence encoding a polypeptide of adenoviral origin is carried by a vector different from said replication-defective adenoviral vector.

14. The method of claim 13, wherein said vector further comprises a transgene.

15. The method of claim 13 or 14, wherein said method comprises introducing in said host cell simultaneously or sequentially (i) said replication-defective adenoviral vector and (ii) said vector comprising said nucleic acid sequence encoding said polypeptide of adenoviral origin.

16. The method of claim 8, wherein said nucleic acid sequence encoding a polypeptide of adenoviral origin is placed under the control of a heterologous promoter selected from the group consisting of constitutive, inducible, tumor-specific and tissue-specific promoters.

17. The method of claim 1, wherein said molecule of adenoviral origin provides a reduction or an inhibition of apoptosis in said host cell.

18. The method of claim 4, wherein said molecule of adenoviral origin provides a reduction or an inhibition of the toxicity induced by said replication-defective adenoviral vector in said host cell and/or an enhancement of the persistence of transgene expression in said host cell.

19. A recombinant adenoviral vector in which the E1 and the E4 regions, and optionally the E3 region, are deleted comprising at least (i) a transgene and (ii) a nucleic acid sequence encoding a functional adenoviral pIX protein, wherein said nucleic acid sequence encoding the functional adenoviral pIX protein is placed under the control of a heterologous promoter and located in said adenoviral vector in a position different from its native location.

20. The recombinant adenoviral vector of claim 19, wherein said nucleic acid sequence encoding the adenoviral pIX protein is located in replacement of the deleted E4 region.

21. The recombinant adenoviral vector of claim 19 or 20, wherein said adenoviral vector further comprises a nucleic acid sequence encoding an adenoviral E4orf3 protein placed under the control of a heterologous promoter.

22. The recombinant adenoviral vector of claim 21, wherein said heterologous promoter is selected from the group consisting of constitutive, inducible, tumor-specific and tissue-specific promoters.

23. A composition comprising the recombinant adenoviral vector of claim 19 or the molecule of adenoviral origin as defined in claim 1, and optionally a pharmaceutically acceptable carrier.

24. A method for reducing or inhibiting one or more cellular activitie(s) dependent on a POD nuclear structure, comprising utilizing therefor the recombinant adenoviral vector of claim 19 or the molecule of adenoviral origin as defined in claim 1.

25. The method of claim 24, wherein said cellular activity is the antiviral cellular activity dependent on said POD nuclear structure in said host cell when infected by a virus.

26. The method of claim 24, wherein said cellular activity is apoptosis in said host cell, optionally when said host cell is infected by a virus.

27. The method of claim 24, wherein said cellular activity is the toxicity induced by a replication-defective adenoviral vector in said host cell and/or an enhancement of a persistence of a transgene expression in said host cell.

28. A replication-competent adenoviral vector, wherein the native adenovirus pIX and/or E4orf3 gene is nonfunctional or deleted.

29. The replication-competent adenoviral vector of claim 28, wherein both native adenovirus pIX and E4orf3 genes are nonfunctional or deleted.

30. The replication-competent adenoviral vector of claim 28 or 29, further comprising a transgene.

31. The replication-competent adenoviral vector of claim 30, wherein said transgene is a suicide gene.

32. The replication-competent adenoviral vector of claim 31, wherein said suicide gene encodes a polypeptide having cytosine deaminase (CDase) and/or a uracile phosphoribosyl transferase (UPRTase) activity.

33. The replication-competent adenoviral vector of claim 32, wherein said suicide gene encodes a fusion polypeptide having both UPRTase and CDase activities.

34. The replication-competent adenoviral vector of claim 30, wherein said transgene is placed under the control of a tumor-specific promoter.

35. A viral particle comprising the replication-competent adenoviral vector of claim 28.

36. A host cell comprising the replication-competent adenoviral vector of claim 28, or infected by the viral particle of claim 35.

37. A composition comprising the replication-competent adenoviral vector of claim 28, the viral particle of claim 35, or the host cell of claim 36.

38. A method of treating a patient suffering from a cancer or a hyperproliferative cell disorder, which comprises administering to said patient a therapeutically effective amount of the replication-competent adenoviral vector of claim 28, or the viral particle of claim 35, or the host cell of claim 36.

39. A method for the preparation of a medicament for the treatment or prevention of a cancer or a hyperproliferative cell disorder by gene therapy, comprising formulating therein the replication-competent adenoviral vector of claim 28, or the viral particle of claim 35, or the host cell of claim 36.

40. A method of enhancing the apoptotic status in a host cell, which comprises introducing in said host cell at least the replication-competent adenoviral vector of claim 28, or the viral particle of claim 35.

41. A method for the preparation of a medicament intended for enhancing apoptosis status in a host cell, comprising formulating therein the replication-competent adenoviral vector of claim 28, or the viral particle of claim 35, or the host cell of claim 36.


SEQUENCE LISTING (2 sequences)

SEQ ID NO: 1 - DNA, 29 bp, Artificial Sequence
  sense primer to clone Ad5 wild-type pIX gene
gaattcgtcgacccatgagcaccaactcg

SEQ ID NO: 2 - DNA, 35 bp, Artificial Sequence
  antisense primer to clone Ad5 wild-type pIX gene
gaattcgatatcttaaaccgcattgggaggggagg